(19) United States
(12) Patent Application Publication
(10) Pub. No.: US 2026/0345835 A1
(43) Pub. Date: Nov. 5, 2026

(54) CELL PERMEANT PARTICLES

(71) Applicant: IONISED TECHNOLOGIES PTY LTD, Newtown, New South Wales (AU)

(72) Inventors: Stuart FRASER, Newtown, New South Wales (AU); Xuege FENG, Northbridge, New South Wales (AU); Behnam AKHAVAN, Blue Bay, New South Wales (AU); Laura HAIDAR, Bexley, New South Wales (AU); Clara Thao Hoang TRAN, Revesby, New South Wales (AU); Badwi Bob BOUMELHEM, Concord, New South Wales (AU); Marcela BILEK, Sutherland, New South Wales (AU)

(21) Appl. No.: 19/118,499

(22) PCT Filed: Sep. 28, 2023
(86) PCT No.: PCT/AU2023/050940

§ 371 Date: Apr. 4, 2025

(30) Foreign Application Priority Data
Oct. 4, 2022 (AU) 2022902871

(51) Int. Cl. A61K 49/00; A61K 49/10; G01N 33/533

(52) U.S. Cl. CPC A61K 49/0054; A61K 49/0091; A61K 49/101; G01N 33/533

(57) ABSTRACT

The present invention relates to cell permeant particles. In particular, the present invention relates to plasma-treated particles or plasma-synthesised particles that are capable of migrating across the membranes of live cells.

### FIELD OF THE INVENTION

The present application claims priority from Australian Provisional Patent Application No. 2022902871 (filed 4 Oct. 2022), the contents of which are incorporated in their entirety herein.

The present invention relates to cell permeant particles. In particular, the present invention relates to plasma-treated particles or plasma-synthesised particles that are capable of migrating across the membranes of live cells. However, it will be appreciated that the invention is not limited to this particular field of use.

### BACKGROUND OF THE INVENTION

Any discussion of the prior art throughout the specification should in no way be considered as an admission that such prior art is widely known or forms part of common general knowledge in the field.

Delivery of particles to cells is currently dependent on uptake via endocytosis or via targeting of cell surface receptors. Endosomal entrapment and subsequent degradation in acidic compartments of the endo/lysosomal pathway means that the particles and any payload (e.g., proteins, peptides, carbohydrates, nucleic acids, small molecules or therapeutic agents) may not reach intracellular locations intact. Additionally, current magnetic activated cell sorting protocols are limited to isolating cells according to cell surface markers/characteristics.

There is a need for particles that are capable of crossing the membrane of live cells.

It is an object of the present invention to overcome or ameliorate at least one of the disadvantages of the prior art, or to provide a useful alternative.

### SUMMARY OF THE INVENTION

It has been surprisingly found that plasma-treated particles or plasma-synthesised particles are lipophilic and capable of migrating across the membranes of live cells. This migration is enhanced by covalently binding a cell permeant agent to the plasma-treated or plasma-synthesised particles.

In a first aspect, the present invention relates to a plasma-treated particle or plasma-synthesised particle to which a cell permeant agent is bound.

In one embodiment, the cell permeant agent is covalently bound to the particle.

In one embodiment, the cell permeant agent binds to a cellular component of interest.

In one embodiment, the cellular component of interest is intracellular.

In one embodiment, the cell permeant agent is lipophilic.

In one embodiment, the cell permeant agent is capable of fluorescing.

In one embodiment, the cell permeant agent is selected from the group consisting of Nile Red, Nile Blue and Mitotracker, Glucose-Iridium complex, carborane coumarin, BODIPY dyes (e.g., BODIPY 505/515), LipiTOX dyes (e.g., LipiTOX Green) and Lipi dyes (e.g., LipiGreen, LipiBlue and LipiRed), or analogues thereof.

In one embodiment, the cell permeant agent is selected from the group consisting of Nile Red, Nile Blue, Mitotracker Green, Glucose-Iridium complex and carborane coumarin, or analogues thereof.

In one embodiment, a payload is bound to the particle for delivery to a cell.

In one embodiment, the payload is covalently bound to the particle.

In one embodiment, the payload is selected from the group consisting of proteins, peptides, carbohydrates, nucleic acids, small molecules and therapeutic agents.

In one embodiment, the payload is a cyclodextrin.

In one embodiment, the payload is beta-cyclodextrin.

In one embodiment, the particle is treated or synthesised by plasma immersion ion implantation (PIII).

In one embodiment, the PIII comprises generating nitrogen plasma.

In one embodiment, the PIII comprises generating nitrogen plasma and accelerating nitrogen ions into the particle.

In one embodiment, the PIII treatment comprises generating argon plasma.

In one embodiment, the PIII treatment comprises generating argon plasma and accelerating argon ions into the particle.

In one embodiment, the plasma is generated by applying pulses.

In one embodiment, the particle is agitated during Pill treatment.

In one embodiment, the plasma-treated particle is a microparticle.

In one embodiment, the plasma-treated particle is a nanoparticle.

In one embodiment, the plasma-treated particle is a paramagnetic particle.

In one embodiment, the paramagnetic particle comprises carbon.

In one embodiment, the paramagnetic particle comprises plastic.

In one embodiment, the paramagnetic particle comprises polystyrene.

In one embodiment, the paramagnetic particle comprises an iron oxide.

In one embodiment, the plasma-synthesised particle is a microparticle.

In one embodiment, the plasma-synthesised particle is a nanoparticle.

In a second aspect, the present invention relates to a method for producing a particle that is capable of migrating across a cell membrane, comprising treating a particle with plasma or synthesising a particle by plasma polymerisation, and binding a cell permeant agent to the particle.

In one embodiment, the cell permeant agent is covalently bound to the particle.

In one embodiment, the cell permeant agent binds to a cellular component of interest.

In one embodiment, the cellular component of interest is intracellular.

In one embodiment, the cell permeant agent is lipophilic.

In one embodiment, the cell permeant agent is capable of fluorescing.

In one embodiment, the cell permeant agent is selected from the group consisting of Nile Red, Nile Blue and Mitotracker, Glucose-Iridium complex, carborane coumarin, BODIPY dyes (e.g., BODIPY 505/515), LipiTOX dyes (e.g., LipiTOX Green) and Lipi dyes (e.g., LipiGreen, LipiBlue and LipiRed), or analogues thereof.

In one embodiment, the cell permeant agent is selected from the group consisting of Nile Red, Nile Blue, Mitotracker Green, Glucose-Iridium complex and carborane coumarin, or analogues thereof.

In one embodiment, a payload is bound to the particle for delivery to a cell.

In one embodiment, the payload is covalently bound to the particle.

In one embodiment, the payload is selected from the group consisting of proteins, peptides, carbohydrates, nucleic acids, small molecules and therapeutic agents.

In one embodiment, the payload is a cyclodextrin.

In one embodiment, the payload is beta-cyclodextrin.

In one embodiment, the particle is treated or synthesised by plasma immersion ion implantation (PIII).

In one embodiment, the PIII comprises generating nitrogen plasma.

In one embodiment, the PIII comprises generating nitrogen plasma and accelerating nitrogen ions into the particle.

In one embodiment, the PIII treatment comprises generating argon plasma.

In one embodiment, the PIII treatment comprises generating argon plasma and accelerating argon ions into the particle.

In one embodiment, the plasma is generated by applying pulses.

In one embodiment, the particle is agitated during PIII treatment.

In one embodiment, the plasma-treated particle is a microparticle.

In one embodiment, the plasma-treated particle is a nanoparticle.

In one embodiment, the plasma-treated particle is a paramagnetic particle.

In one embodiment, the paramagnetic particle comprises carbon.

In one embodiment, the paramagnetic particle comprises plastic.

In one embodiment, the paramagnetic particle comprises polystyrene.

In one embodiment, the paramagnetic particle comprises an iron oxide.

In one embodiment, the plasma-synthesised particle is a microparticle.

In one embodiment, the plasma-synthesised particle is a nanoparticle.

In a third aspect, the present invention relates to a particle produced by the method of the second aspect.

In a fourth aspect, the present invention relates to a method for producing a lipophilic particle, comprising treating a particle with plasma or synthesising a particle by plasma polymerisation.

In one embodiment, a cell permeant agent is bound to the particle.

In one embodiment, the cell permeant agent is covalently bound to the particle.

In one embodiment, the cell permeant agent binds to a cellular component of interest.

In one embodiment, the cellular component of interest is intracellular.

In one embodiment, the cell permeant agent is lipophilic.

In one embodiment, the cell permeant agent is capable of fluorescing.

In one embodiment, the cell permeant agent is selected from the group consisting of Nile Red, Nile Blue and Mitotracker, Glucose-Iridium complex, carborane coumarin, BODIPY dyes (e.g., BODIPY 505/515), LipiTOX dyes (e.g., LipiTOX Green) and Lipi dyes (e.g., LipiGreen, LipiBlue and LipiRed), or analogues thereof.

In one embodiment, the cell permeant agent is selected from the group consisting of Nile Red, Nile Blue, Mitotracker Green, Glucose-Iridium complex and carborane coumarin, or analogues thereof.

In one embodiment, a payload is bound to the particle for delivery to a cell.

In one embodiment, the payload is covalently bound to the particle.

In one embodiment, the payload is selected from the group consisting of proteins, peptides, carbohydrates, nucleic acids, small molecules and therapeutic agents.

In one embodiment, the payload is a cyclodextrin.

In one embodiment, the payload is beta-cyclodextrin.

In one embodiment, the particle is treated or synthesised by plasma immersion ion implantation (PIII).

In one embodiment, the PIII comprises generating nitrogen plasma.

In one embodiment, the PIII comprises generating nitrogen plasma and accelerating nitrogen ions into the particle.

In one embodiment, the PIII treatment comprises generating argon plasma.

In one embodiment, the PIII treatment comprises generating argon plasma and accelerating argon ions into the particle.

In one embodiment, the plasma is generated by applying pulses.

In one embodiment, the particle is agitated during PIII treatment.

In one embodiment, the plasma-treated particle is a microparticle.

In one embodiment, the plasma-treated particle is a nanoparticle.

In one embodiment, the plasma-treated particle is a paramagnetic particle.

In one embodiment, the paramagnetic particle comprises carbon.

In one embodiment, the paramagnetic particle comprises plastic.

In one embodiment, the paramagnetic particle comprises polystyrene.

In one embodiment, the paramagnetic particle comprises an iron oxide.

In one embodiment, the plasma-synthesised particle is a microparticle.

In one embodiment, the plasma-synthesised particle is a nanoparticle.

In a fifth aspect, the present invention relates to a particle produced by the method of the fourth aspect.

In a sixth aspect, the present invention relates to a method for identifying a cell based on a cellular component of interest, the method comprising administering a plasma-treated particle or plasma-synthesised particle to the cell, wherein a cell permeant agent is bound to the particle, and wherein the cell permeant agent binds to the cellular component of interest.

In one embodiment, the cell permeant agent is covalently bound to the particle.

In one embodiment, the cellular component of interest is intracellular.

In one embodiment, the cell permeant agent is lipophilic.

In one embodiment, the cell permeant agent is capable of fluorescing.

In one embodiment, the agent is selected from the group consisting of Nile Red, Nile Blue and Mitotracker, Glucose-Iridium complex, carborane coumarin, BODIPY dyes (e.g., BODIPY 505/515), LipiTOX dyes (e.g., LipiTOX Green) and Lipi dyes (e.g., LipiGreen, LipiBlue and LipiRed), or analogues thereof.

In one embodiment, the agent is selected from the group consisting of Nile Red, Nile Blue, Mitotracker Green, Glucose-Iridium complex and carborane coumarin, or analogues thereof.

In one embodiment, a payload is bound to the particle for delivery to the cell.

In one embodiment, the payload is covalently bound to the particle.

In one embodiment, the payload is selected from the group consisting of proteins, peptides, carbohydrates, nucleic acids, small molecules and therapeutic agents.

In one embodiment, the payload is a cyclodextrin.

In one embodiment, the payload is beta-cyclodextrin.

In one embodiment, the particle is treated or synthesised by plasma immersion ion implantation (PIII).

In one embodiment, the PIII comprises generating nitrogen plasma.

In one embodiment, the PIII comprises generating nitrogen plasma and accelerating nitrogen ions into the particle.

In one embodiment, the PIII treatment comprises generating argon plasma.

In one embodiment, the PIII treatment comprises generating argon plasma and accelerating argon ions into the particle.

In one embodiment, the plasma is generated by applying pulses.

In one embodiment, the particle is agitated during PIII treatment.

In one embodiment, the plasma-treated particle is a microparticle.

In one embodiment, the plasma-treated particle is a nanoparticle.

In one embodiment, the plasma-treated particle is a paramagnetic particle.

In one embodiment, the paramagnetic particle comprises carbon.

In one embodiment, the paramagnetic particle comprises plastic.

In one embodiment, the paramagnetic particle comprises polystyrene.

In one embodiment, the paramagnetic particle comprises an iron oxide.

In one embodiment, the plasma-synthesised particle is a microparticle.

In one embodiment, the plasma-synthesised particle is a nanoparticle.

In one embodiment, the cell is part of a population of cells.

In one embodiment, the method is performed in vitro.

In one embodiment, the method is performed in vivo.

In one embodiment, the method is performed in vivo in a non-human mammal.

In a seventh aspect, the present invention relates to a method of delivering a payload to a cell, the method comprising administering a plasma-treated particle or plasma-synthesised particle to the cell, wherein a cell permeant agent and the payload are bound to the particle.

In one embodiment, the cell permeant agent is covalently bound to the particle.

In one embodiment, the cell permeant agent binds to a cellular component of interest.

In one embodiment, the cellular component of interest is intracellular.

In one embodiment, the cell permeant agent is lipophilic.

In one embodiment, the cell permeant agent is capable of fluorescing.

In one embodiment, the cell permeant agent is selected from the group consisting of Nile Red, Nile Blue and Mitotracker, Glucose-Iridium complex, carborane coumarin, BODIPY dyes (e.g., BODIPY 505/515), LipiTOX dyes (e.g., LipiTOX Green) and Lipi dyes (e.g., LipiGreen, LipiBlue and LipiRed), or analogues thereof.

In one embodiment, the cell permeant agent is selected from the group consisting of Nile Red, Nile Blue, Mitotracker Green, Glucose-Iridium complex and carborane coumarin, or analogues thereof.

In one embodiment, the payload is covalently bound to the particle.

In one embodiment, the payload is selected from the group consisting of proteins, peptides, carbohydrates, nucleic acids, small molecules and therapeutic agents.

In one embodiment, the payload is a cyclodextrin.

In one embodiment, the payload is beta-cyclodextrin.

In one embodiment, the particle is treated or synthesised by plasma immersion ion implantation (PIII).

In one embodiment, the PIII comprises generating nitrogen plasma.

In one embodiment, the PIII comprises generating nitrogen plasma and accelerating nitrogen ions into the particle.

In one embodiment, the PIII treatment comprises generating argon plasma.

In one embodiment, the PIII treatment comprises generating argon plasma and accelerating argon ions into the particle.

In one embodiment, the plasma is generated by applying pulses.

In one embodiment, the particle is agitated during PIII treatment.

In one embodiment, the plasma-treated particle is a microparticle.

In one embodiment, the plasma-treated particle is a nanoparticle.

In one embodiment, the plasma-treated particle is a paramagnetic particle.

In one embodiment, the paramagnetic particle comprises carbon.

In one embodiment, the paramagnetic particle comprises plastic.

In one embodiment, the paramagnetic particle comprises polystyrene.

In one embodiment, the paramagnetic particle comprises an iron oxide.

In one embodiment, the plasma-synthesised particle is a microparticle.

In one embodiment, the plasma-synthesised particle is a nanoparticle.

In one embodiment, the cell is part of a population of cells.

In one embodiment, the method is performed in vitro.

In one embodiment, the method is performed in vivo.

In one embodiment, the method is performed in vivo in a non-human mammal.

In an eighth aspect, the present invention relates to a method of magnetically sorting a cell based on a cellular component of interest, the method comprising:
administering a plasma-treated paramagnetic microparticle to the cell, wherein a cell permeant agent is bound to the microparticle, and wherein the cell permeant agent binds to the cellular component of interest; and
applying a magnetic field to the cell.

In one embodiment, the cell permeant agent is covalently bound to the particle.

In one embodiment, the cellular component of interest is intracellular.

In one embodiment, the cell permeant agent is lipophilic.

In one embodiment, the cell permeant agent is capable of fluorescing.

In one embodiment, the cell permeant agent is selected from the group consisting of Nile Red, Nile Blue and Mitotracker, Glucose-Iridium complex, carborane coumarin, BODIPY dyes (e.g., BODIPY 505/515), LipiTOX dyes (e.g., LipiTOX Green) and Lipi dyes (e.g., LipiGreen, LipiBlue and LipiRed), or analogues thereof.

In one embodiment, the cell permeant agent is selected from the group consisting of Nile Red, Nile Blue, Mitotracker Green, Glucose-Iridium complex and carborane coumarin, or analogues thereof.

In one embodiment, a payload is bound to the particle for delivery to the cell.

In one embodiment, the payload is covalently bound to the particle.

In one embodiment, the payload is selected from the group consisting of proteins, peptides, carbohydrates, nucleic acids, small molecules and therapeutic agents.

In one embodiment, the payload is a cyclodextrin.

In one embodiment, the payload is beta-cyclodextrin.

In one embodiment, the particle is treated or synthesised by plasma immersion ion implantation (PIII).

In one embodiment, the PIII comprises generating nitrogen plasma.

In one embodiment, the PIII comprises generating nitrogen plasma and accelerating nitrogen ions into the particle.

In one embodiment, the PIII treatment comprises generating argon plasma.

In one embodiment, the PIII treatment comprises generating argon plasma and accelerating argon ions into the particle.

In one embodiment, the plasma is generated by applying pulses.

In one embodiment, the particle is agitated during PIII treatment.

In one embodiment, the plasma-treated particle is a microparticle.

In one embodiment, the plasma-treated particle is a nanoparticle.

In one embodiment, the plasma-treated particle is a paramagnetic particle.

In one embodiment, the paramagnetic particle comprises carbon.

In one embodiment, the paramagnetic particle comprises plastic.

In one embodiment, the paramagnetic particle comprises polystyrene.

In one embodiment, the paramagnetic particle comprises an iron oxide.

In one embodiment, the plasma-synthesised particle is a microparticle.

In one embodiment, the plasma-synthesised particle is a nanoparticle.

In one embodiment, the cell is part of a population of cells.

In one embodiment, the method is performed in vitro.

In one embodiment, the method is performed in vivo.

In one embodiment, the method is performed in vivo in a non-human mammal.

In a ninth aspect, the present invention relates to a method of treating a disease in a subject, comprising administering to the subject a plasma-treated particle or plasma-synthesised particle, wherein a cell permeant agent is bound to the particle.

In one embodiment, the cell permeant agent is covalently bound to the particle.

In one embodiment, the cell permeant agent binds to a cellular component of interest.

In one embodiment, the cellular component of interest is intracellular.

In one embodiment, the cell permeant agent is lipophilic.

In one embodiment, the cell permeant agent is capable of fluorescing.

In one embodiment, the cell permeant agent is selected from the group consisting of Nile Red, Nile Blue and Mitotracker, Glucose-Iridium complex, carborane coumarin, BODIPY dyes (e.g., BODIPY 505/515), LipiTOX dyes (e.g., LipiTOX Green) and Lipi dyes (e.g., LipiGreen, LipiBlue and LipiRed), or analogues thereof.

In one embodiment, the cell permeant agent is selected from the group consisting of Nile Red, Nile Blue, Mitotracker Green, Glucose-Iridium complex and carborane coumarin, or analogues thereof.

In one embodiment, a payload is bound to the particle for delivery to a cell.

In one embodiment, the therapeutic agent is covalently bound to the particle.

In one embodiment, the payload is selected from the group consisting of proteins, peptides, carbohydrates, nucleic acids, small molecules and therapeutic agents.

In one embodiment, the therapeutic agent is a cyclodextrin.

In one embodiment, the therapeutic agent is beta-cyclodextrin.

In one embodiment, the particle is treated or synthesised by plasma immersion ion implantation (PIII).

In one embodiment, the PIII comprises generating nitrogen plasma.

In one embodiment, the PIII comprises generating nitrogen plasma and accelerating nitrogen ions into the particle.

In one embodiment, the PIII treatment comprises generating argon plasma.

In one embodiment, the PIII treatment comprises generating argon plasma and accelerating argon ions into the particle.

In one embodiment, the plasma is generated by applying pulses.

In one embodiment, the particle is agitated during PIII treatment.

In one embodiment, the plasma-treated particle is a microparticle.

In one embodiment, the plasma-treated particle is a nanoparticle.

In one embodiment, the plasma-treated particle is a paramagnetic particle.

In one embodiment, the paramagnetic particle comprises carbon.

In one embodiment, the paramagnetic particle comprises plastic.

In one embodiment, the paramagnetic particle comprises polystyrene.

In one embodiment, the paramagnetic particle comprises an iron oxide.

In one embodiment, the plasma-synthesised particle is a microparticle.

In one embodiment, the plasma-synthesised particle is a nanoparticle.

In one embodiment, the disease is cancer.

In one embodiment, the disease is cancer and the therapeutic agent is beta-cyclodextrin.

In a tenth aspect, the present invention relates to use of a plasma-treated particle or plasma-synthesised particle for the manufacture of a medicament for the treatment of a disease, wherein a cell permeant agent is bound to the particle.

In one embodiment, the cell permeant agent is covalently bound to the particle.

In one embodiment, the cell permeant agent binds to a cellular component of interest.

In one embodiment, the cellular component of interest is intracellular.

In one embodiment, the cell permeant agent is lipophilic.

In one embodiment, the cell permeant agent is capable of fluorescing.

In one embodiment, the cell permeant agent is selected from the group consisting of Nile Red, Nile Blue and Mitotracker, Glucose-Iridium complex, carborane coumarin, BODIPY dyes (e.g., BODIPY 505/515), LipiTOX dyes (e.g., LipiTOX Green) and Lipi dyes (e.g., LipiGreen, LipiBlue and LipiRed), or analogues thereof.

In one embodiment, the cell permeant agent is selected from the group consisting of Nile Red, Nile Blue, Mitotracker Green, Glucose-Iridium complex and carborane coumarin, or analogues thereof.

In one embodiment, a payload is bound to the particle for delivery to a cell.

In one embodiment, the therapeutic agent is covalently bound to the particle.

In one embodiment, the payload is selected from the group consisting of proteins, peptides, carbohydrates, nucleic acids, small molecules and therapeutic agents.

In one embodiment, the therapeutic agent is a cyclodextrin.

In one embodiment, the therapeutic agent is beta-cyclodextrin.

In one embodiment, the particle is treated or synthesised by plasma immersion ion implantation (PIII).

In one embodiment, the PIII comprises generating nitrogen plasma.

In one embodiment, the PIII comprises generating nitrogen plasma and accelerating nitrogen ions into the particle.

In one embodiment, the PIII treatment comprises generating argon plasma.

In one embodiment, the PIII treatment comprises generating argon plasma and accelerating argon ions into the particle.

In one embodiment, the plasma is generated by applying pulses.

In one embodiment, the particle is agitated during PIII treatment.

In one embodiment, the plasma-treated particle is a microparticle.

In one embodiment, the plasma-treated particle is a nanoparticle.

In one embodiment, the plasma-treated particle is a paramagnetic particle.

In one embodiment, the paramagnetic particle comprises carbon.

In one embodiment, the paramagnetic particle comprises plastic.

In one embodiment, the paramagnetic particle comprises polystyrene.

In one embodiment, the paramagnetic particle comprises an iron oxide.

In one embodiment, the plasma-synthesised particle is a microparticle.

In one embodiment, the plasma-synthesised particle is a nanoparticle.

In one embodiment, the disease is cancer.

In one embodiment, the disease is cancer and the therapeutic agent is beta-cyclodextrin.

In an eleventh aspect, the present invention provides a composition comprising a plasma-treated particle or plasma-synthesised particle for use in the treatment of a disease, wherein a cell permeant agent is bound to the particle.

In one embodiment, the cell permeant agent is covalently bound to the particle.

In one embodiment, the cell permeant agent binds to a cellular component of interest.

In one embodiment, the cellular component of interest is intracellular.

In one embodiment, the cell permeant agent is lipophilic.

In one embodiment, the cell permeant agent is capable of fluorescing.

In one embodiment, the cell permeant agent is selected from the group consisting of Nile Red, Nile Blue and Mitotracker, Glucose-Iridium complex, carborane coumarin, BODIPY dyes (e.g., BODIPY 505/515), LipiTOX dyes (e.g., LipiTOX Green) and Lipi dyes (e.g., LipiGreen, LipiBlue and LipiRed), or analogues thereof.

In one embodiment, the cell permeant agent is selected from the group consisting of Nile Red, Nile Blue, Mitotracker Green, Glucose-Iridium complex and carborane coumarin, or analogues thereof.

In one embodiment, a payload is bound to the particle for delivery to a cell.

In one embodiment, the therapeutic agent is covalently bound to the particle.

In one embodiment, the payload is selected from the group consisting of proteins, peptides, carbohydrates, nucleic acids, small molecules and therapeutic agents.

In one embodiment, the therapeutic agent is a cyclodextrin.

In one embodiment, the therapeutic agent is beta-cyclodextrin.

In one embodiment, the particle is treated or synthesised by plasma immersion ion implantation (PIII).

In one embodiment, the PIII comprises generating nitrogen plasma.

In one embodiment, the PIII comprises generating nitrogen plasma and accelerating nitrogen ions into the particle.

In one embodiment, the PIII treatment comprises generating argon plasma.

In one embodiment, the PIII treatment comprises generating argon plasma and accelerating argon ions into the particle.

In one embodiment, the plasma is generated by applying pulses.

In one embodiment, the particle is agitated during PIII treatment.

In one embodiment, the plasma-treated particle is a microparticle.

In one embodiment, the plasma-treated particle is a nanoparticle.

In one embodiment, the plasma-treated particle is a paramagnetic particle.

In one embodiment, the paramagnetic particle comprises carbon.

In one embodiment, the paramagnetic particle comprises plastic.

In one embodiment, the paramagnetic particle comprises polystyrene.

In one embodiment, the paramagnetic particle comprises an iron oxide.

In one embodiment, the plasma-synthesised particle is a microparticle.

In one embodiment, the plasma-synthesised particle is a nanoparticle.

In one embodiment, the disease is cancer.

In one embodiment, the disease is cancer and the therapeutic agent is beta-cyclodextrin.

In a twelfth aspect, the present invention provides a pharmaceutical composition comprising a plasma-treated particle or plasma-synthesised particle, wherein a cell permeant agent is bound to the particle.

In one embodiment, the cell permeant agent is covalently bound to the particle.

In one embodiment, the cell permeant agent binds to a cellular component of interest.

In one embodiment, the cellular component of interest is intracellular.

In one embodiment, the cell permeant agent is lipophilic.

In one embodiment, the cell permeant agent is capable of fluorescing.

In one embodiment, the cell permeant agent is selected from the group consisting of Nile Red, Nile Blue and Mitotracker, Glucose-Iridium complex, carborane coumarin, BODIPY dyes (e.g., BODIPY 505/515), LipiTOX dyes (e.g., LipiTOX Green) and Lipi dyes (e.g., LipiGreen, LipiBlue and LipiRed), or analogues thereof.

In one embodiment, the cell permeant agent is selected from the group consisting of Nile Red, Nile Blue, Mitotracker Green, Glucose-Iridium complex and carborane coumarin, or analogues thereof.

In one embodiment, a payload is bound to the particle for delivery to a cell.

In one embodiment, the therapeutic agent is covalently bound to the particle.

In one embodiment, the payload is selected from the group consisting of proteins, peptides, carbohydrates, nucleic acids, small molecules and therapeutic agents.

In one embodiment, the therapeutic agent is a cyclodextrin.

In one embodiment, the therapeutic agent is beta-cyclodextrin.

In one embodiment, the particle is treated or synthesised by plasma immersion ion implantation (PIII).

In one embodiment, the PIII comprises generating nitrogen plasma.

In one embodiment, the PIII comprises generating nitrogen plasma and accelerating nitrogen ions into the particle.

In one embodiment, the PIII treatment comprises generating argon plasma.

In one embodiment, the PIII treatment comprises generating argon plasma and accelerating argon ions into the particle.

In one embodiment, the plasma is generated by applying pulses.

In one embodiment, the particle is agitated during PIII treatment.

In one embodiment, the plasma-treated particle is a microparticle.

In one embodiment, the plasma-treated particle is a nanoparticle.

In one embodiment, the plasma-treated particle is a paramagnetic particle.

In one embodiment, the paramagnetic particle comprises carbon.

In one embodiment, the paramagnetic particle comprises plastic.

In one embodiment, the paramagnetic particle comprises polystyrene.

In one embodiment, the paramagnetic particle comprises an iron oxide.

In one embodiment, the plasma-synthesised particle is a microparticle.

In one embodiment, the plasma-synthesised particle is a nanoparticle.

In one embodiment, the disease is cancer.

In one embodiment, the disease is cancer and the therapeutic agent is beta-cyclodextrin.

In a thirteenth aspect, the present invention provides a pharmaceutical composition or a diagnostic composition comprising a plasma-treated particle or plasma-synthesised particle, wherein a cell permeant agent is bound to the particle.

In one embodiment, the cell permeant agent is covalently bound to the particle.

In one embodiment, the cell permeant agent binds to a cellular component of interest.

In one embodiment, the cellular component of interest is intracellular.

In one embodiment, the cell permeant agent is lipophilic.

In one embodiment, the cell permeant agent is capable of fluorescing.

In one embodiment, the cell permeant agent is selected from the group consisting of Nile Red, Nile Blue and Mitotracker, Glucose-Iridium complex, carborane coumarin, BODIPY dyes (e.g., BODIPY 505/515), LipiTOX dyes (e.g., LipiTOX Green) and Lipi dyes (e.g., LipiGreen, LipiBlue and LipiRed), or analogues thereof.

In one embodiment, the cell permeant agent is selected from the group consisting of Nile Red, Nile Blue, Mitotracker Green, Glucose-Iridium complex and carborane coumarin, or analogues thereof.

In one embodiment, a payload is bound to the particle for delivery to a cell.

In one embodiment, the payload is selected from the group consisting of proteins, peptides, carbohydrates, nucleic acids, small molecules and therapeutic agents.

In one embodiment, the therapeutic agent is a cyclodextrin.

In one embodiment, the therapeutic agent is beta-cyclodextrin.

In one embodiment, the therapeutic agent is covalently bound to the particle.

In one embodiment, the disease is cancer.

In one embodiment, the particle is treated or synthesised by plasma immersion ion implantation (PIII).

In one embodiment, the PIII comprises generating nitrogen plasma.

In one embodiment, the PIII comprises generating nitrogen plasma and accelerating nitrogen ions into the particle.

In one embodiment, the PIII treatment comprises generating argon plasma.

In one embodiment, the PIII treatment comprises generating argon plasma and accelerating argon ions into the particle.

In one embodiment, the plasma is generated by applying pulses.

In one embodiment, the particle is agitated during PIII treatment.

In one embodiment, the plasma-treated particle is a microparticle.

In one embodiment, the plasma-treated particle is a nanoparticle.

In one embodiment, the plasma-treated particle is a paramagnetic particle.

In one embodiment, the paramagnetic particle comprises carbon.

In one embodiment, the paramagnetic particle comprises plastic.

In one embodiment, the paramagnetic particle comprises polystyrene.

In one embodiment, the paramagnetic particle comprises an iron oxide.

In one embodiment, the plasma-synthesised particle is a microparticle.

In one embodiment, the plasma-synthesised particle is a nanoparticle.

In a fourteenth aspect, the present invention provides a method of diagnosis of a disease or condition in a subject, comprising administering to the subject a plasma-treated particle or plasma-synthesised particle, wherein a cell permeant agent is bound to the particle.

In one embodiment, the cell permeant agent is covalently bound to the particle.

In one embodiment, the cell permeant agent binds to a cellular component of interest.

In one embodiment, the cellular component of interest is intracellular.

In one embodiment, the cell permeant agent is lipophilic.

In one embodiment, the cell permeant agent is capable of fluorescing.

In one embodiment, the cell permeant agent is selected from the group consisting of Nile Red, Nile Blue and Mitotracker, Glucose-Iridium complex, carborane coumarin, BODIPY dyes (e.g., BODIPY 505/515), LipiTOX dyes (e.g., LipiTOX Green) and Lipi dyes (e.g., LipiGreen, LipiBlue and LipiRed), or analogues thereof.

In one embodiment, the cell permeant agent is selected from the group consisting of Nile Red, Nile Blue, Mitotracker Green, Glucose-Iridium complex and carborane coumarin, or analogues thereof.

In one embodiment, a payload is bound to the particle for delivery to a cell.

In one embodiment, the payload is selected from the group consisting of proteins, peptides, carbohydrates, nucleic acids, small molecules and therapeutic agents.

In one embodiment, the therapeutic agent is a cyclodextrin.

In one embodiment, the therapeutic agent is beta-cyclodextrin.

In one embodiment, the therapeutic agent is covalently bound to the particle.

In one embodiment, the disease is cancer.

In one embodiment, the particle is treated or synthesised by plasma immersion ion implantation (PIII).

In one embodiment, the PIII comprises generating nitrogen plasma.

In one embodiment, the PIII comprises generating nitrogen plasma and accelerating nitrogen ions into the particle.

In one embodiment, the PIII treatment comprises generating argon plasma.

In one embodiment, the PIII treatment comprises generating argon plasma and accelerating argon ions into the particle.

In one embodiment, the plasma is generated by applying pulses.

In one embodiment, the particle is agitated during PIII treatment.

In one embodiment, the plasma-treated particle is a microparticle.

In one embodiment, the plasma-treated particle is a nanoparticle.

In one embodiment, the plasma-treated particle is a paramagnetic particle.

In one embodiment, the paramagnetic particle comprises carbon.

In one embodiment, the paramagnetic particle comprises plastic.

In one embodiment, the paramagnetic particle comprises polystyrene.

In one embodiment, the paramagnetic particle comprises an iron oxide.

In one embodiment, the plasma-synthesised particle is a microparticle.

In one embodiment, the plasma-synthesised particle is a nanoparticle.

### BRIEF DESCRIPTION OF THE DRAWINGS

FIG. 1: Plasma immersion ion implantation (PIII) treatment generates two distinct magnetic particle populations. (A) Schematic diagram depicting the plasma immersion ion implantation (PIII) system used for treating microparticles. (B) Images of glass tube used in the PIII treatment with two different locations of copper electrode. The magenta glow is the nitrogen plasma. The electrode was located either at the base of the glass tube for 5 cycles then along the side for another 5 cycles (Treatment 1); or 10 cycles along the side of the tube (Treatment 2); or 10 cycles at the base of the tube (Treatment 3). (C) Left: two types of microparticles were separated after the PIII treatment: particles adhered to the wall of the tube (“walls particles”, within the black brackets) and particles associated with glass beads (“free particles”, white star). (C) Right: The free particles can be easily removed onto the weigh boat (white star) whereas the wall particles remain adhered to the wall and need to be suspended in ddH₂O. (D) Comparison of the number of wall particles (dark grey) and free particles (light grey) generated by each treatment. Data is shown as mean number counted by hemocytometer; error bars indicate SD. *indicates p value<0.05, **indicates p value<0.01, as determined by students t-test using GraphPad Prism.

FIG. 2: PIII treatment induces violet-range autofluorescence on microparticles. (A) Flow cytometry data obtained from each fluorescent channel (FL) of the 10-colour Becton Dickinson Gallios flow cytometer in descending order; untreated, Treatment 1 wall particles, Treatment 1 free particles, Treatment 2 wall particles, Treatment 2 free particles, Treatment 3 wall particles and Treatment 3 free particles. (B) Mean fluorescence intensity (MFI) of wall particle (left) or free particle (right) in FL10 shows that PIII treatment induces autofluorescence. Un=untreated, Tr1=Treatment 1, Tr2=Treatment 2, Tr3=Treatment 3. Data presented are the average from 3 independent experiments with SD, *p value<=0.05, **p value<=0.01, ***p<=0.001 as determined by two-way ANOVA using GraphPad Prism. (C) Confocal imaging of untreated (top row) and plasma-treated particles (bottom row) with non-conjugated plasma-treated MPs (left column), Nile Red (NR)-conjugated plasma-treated microparticles (middle column) or Nile Blue (NB)-conjugated plasma-treated microparticles (right column). Images were taken using a Zeiss LSM 800 confocal microscope with a 63× oil immersion objective and analysed using the Zen Blue software package. Images are pseudocoloured using ImageJ: Nile Red (magenta), Nile Blue (cyan) and violet channel autofluorescence (yellow) were excited at 488 nm, 660 nm and 405 nm respectively. Images shown are a merge of brightfield and all 3 fluorescent channels (405 nm, 488 nm and 660 nm excitation).

FIG. 3: Flow cytometric analysis shows covalent attachment of Nile Red to plasma-treated microparticles. (A) Fluorescence intensities from FL2 (488 nm) and FL6 (640 nm) measured from unstained samples, plasma-treated microparticles without SDS wash, plasma-treated microparticles with SDS wash at RT and plasma-treated microparticles with SDS wash at 70 °C with or without Nile Red (NR) conjugation. (B) Comparison of mean fluorescence intensities measured using FL2 and FL6 channels from three independent experiments for wall (left panels) and free particles (right panels). Tr1=treatment 1; Tr2=treatment 2; Tr3=treatment 3. Black columns indicate unstained samples, solid red indicates ddH₂O (No SDS wash) samples, red with white dots indicates RT 5% SDS samples and red with white hashes indicates 70 °C SDS wash samples. Data show MFI for 3 independent experiments with SD. NS=no statistically significantly difference, *p value<=0.05, **p value<=0.01, ***p<=0.001 as determined by two-way ANOVA using GraphPad Prism. (C) Mean fluorescence intensities from FL6 channel of non-conjugated plasma-treated microparticles, non-conjugated plasma-treated microparticles with SDS wash at 70 °C, Nile Blue (NB)-conjugated plasma-treated microparticles and NB-conjugated plasma-treated microparticle with SDS wash at 70 °C. (D) Flow cytometry data showing fluorescence intensities in FL6 of non-conjugated plasma-treated microparticles, non-conjugated plasma-treated microparticles with SDS wash, NB-conjugated plasma-treated microparticles, and NB-conjugated plasma-treated microparticles with SDS wash.

FIG. 4: Plasma treatment and Nile Red or Nile Blue conjugation changes size and surface charge of microparticles. (A) Dynamic light scattering analysis compares average diameter of microparticles before and after plasma treatment and Nile Red or Nile Blue conjugation. The average size of plasma-treated microparticles altered according to the dispersion solution (pH7.4 buffer or ddH₂O). (B) Comparison of zeta potential of wall particles and free particles, showing wall particles are more positive than free particles at low pH. (C) Comparison of zeta potential of untreated and plasma-treated microparticles in different buffers and in ddH₂O. Plasma-treated microparticles conjugated to Nile Blue were more positive in 5 mM pH7.4 buffer and in ddH₂O. (D) Zeta potential measurement showing SDS wash shifts surface charge towards more negative on all plasma-treated microparticles.

FIG. 5: Plasma treatment increases lipophilicity of microparticles. (A) Phase separation after mixing an equal volume of 1-octanol and ddH 2O containing microparticles. Images were taken after 1 min. Water partitions to the bottom while 1-octanol partitions on top. Left panels show untreated microparticles and right panels show plasma-treated microparticles. Top panels show microparticles incubated with Nile Red while lower panels show microparticles incubated with Nile Blue. Plasma-treated microparticles all settled at the interface of the 1-octanol: water mixture regardless of conjugation. (B) Fluorescent microscopy of Nile-Red conjugated plasma-treated microparticles excited at 540 nm. Nile Red-conjugated untreated microparticles which partitioned to the lower aqueous layer showed no fluorescence (left panels). In contrast, Nile Red-conjugated plasma-treated microparticles collected at the 1-octanol: water interface showed intense fluorescence and clustered around the edge of 1-octanol droplets (right panels).

FIG. 6: Lipophilic probe-conjugated plasma-treated microparticles target specific locations within live cells. 3T3-L1 fibroblasts were differentiated into adipocytes, incubated with microparticles, fixed, stained and imaged by confocal microscopy. Images are pseudocoloured using ImageJ. Yellow indicates phalloidin, detecting F-actin; grey-scale indicates carborane coumarin, a probe which acts similar to Nile Red but fluoresces in the blue range; magenta indicates Nile Red and cyan indicates Nile Blue. (A) Adipocytes without microparticles, (B) Adipocytes with non-conjugated untreated microparticles or (C) adipocytes with non-conjugated plasma-treated microparticles. Images in (A, B and C) are merged of all channels. (D and E) show NR-conjugated untreated microparticles and plasma-treated microparticles respectively following incubation with adipocytes. The large panel on the right shows merged image of phalloidin, NR (ex: 488 nm: em: 575 nm) and NB (ex: 640 nm: em: 660 nm) in cells incubated with untreated NR-conjugated microparticles s (10 MPs/cell). Smaller inset panels on the left show individual channels for NR (em: 575 nm) and NR (em: 660 nm). (F and G) show NB-conjugated untreated microparticles and plasma-treated microparticles respectively following incubation with adipocytes. The large panel on the right shows merged image of phalloidin, carborane coumarin (ex: 405 nm: em: 550 nm) and Nile Blue (660 nm) in cells incubated 10 MPs/cell. The smaller inset panels on the left show individual channels for carborane coumarin and Nile Blue. (H) shows optical zoom images of the 660 nm fluorescent signal alone. Images (left to right) show: NR-conjugated untreated microparticles, NR-conjugated plasma-treated microparticles, NB-conjugated untreated microparticles and NB-conjugated plasma-treated microparticles. NR-conjugated microparticles are indicated by white arrows and more readily observed in plasma-treated microparticles incubated adipocytes. Images were taken using a Zeiss LSM 800 confocal microscope with a 40× oil immersion objective and analysed using the Zen Blue software package.

FIG. 7: Histochemomagnetic separation of adipocytes by lipid content targeting. (A) Confocal microscopic imaging of the magnetic fraction (left panels) and non-magnetic fraction (right panels) after 3T3-L1-differentiated adipocytes were incubated with Nile Red-conjugated plasma-treated microparticles and separated using a magnet. Insets in top right corner of non-magnetic fraction are brightfield images of cells present in non-magnetic fraction added as fluorescent signal for NR and NB is very low in the non-magnetic fraction. Top images were captured using 20× objective while lower images were captured using 63× oil immersion objective. The brightness and contrast of the images from the non-magnetic fraction were significantly enhanced using Affinity photo in an attempt to reveal any fluorescent signal however very little was evident. (B) Representative flow cytometric profiles from the magnetic (magnetic) or non-magnetic (N/M) fraction from untreated microparticles or plasma-treated particles. Either 10 (10/cell) or 5 (5/cell) particles were used per cell. FL2 (575 nm) and FL6 (660 nm) channels are shown. (C and D) Quantification of MFI shows a significant increase in NR FL6 (660 nm) fluorescence in the magnetic fraction compared to the non-magnetic fraction only with plasma-treated microparticles. Data shows mean with SD from 3 independent experiments. (E) Confocal microscopic imaging of the magnetic fraction (left panels) and non-magnetic fraction (right panels) from 3T3-L1-differentiated adipocytes incubated with Nile Blue-conjugated plasma-treated microparticles. Top images=20× objective, lower images=x63 objective using the Zeiss LMS800 confocal microscope. The exposure of the images from the non-magnetic fraction was significantly enhanced using Affinity photo in an attempt to reveal any fluorescent signal however very little was evident. (F) Representative flow cytometric profiles from the magnetic or non-magnetic fractions from untreated particles conjugated to Nile Blue (cyan) or plasma-treated particles. Ten particles were used per cell (10/cell). (G) MFI shows there is a significant increase in NB FL6 (660 nm) fluorescence in magnetic compared to the non-magnetic fractions in 3T3-LI adipocytes magnetically selected with Nile Blue-conjugated plasma-treated microparticles. Data are MFI from 3 independent experiments showing SD. NS=not significant, *p value<=0.05 as assessed by two-way ANOVA using GraphPad Prism. (G)

FIG. 8: Histochemomagnetic separation of adrenal gland and adipose cells targeted via lipid content. (A) Single cell suspensions of young male Wistar rat adrenal glands were incubated in a 37 °C, 5% CO2 incubator with 10 Nile Red-conjugated plasma-treated microparticles per cell for 60 mins. Phase contrast images were taken of the adrenal gland cell suspensions with Nile Red-conjugated plasma-treated microparticles during the incubation period (left panel), of the magnetic fraction (middle panel) and the non-magnetic fraction. Nile Red-conjugated plasma-treated microparticles can be observed within cells during the incubation and are evident following magnetic selection (black arrowheads). The non-magnetic fraction consisted of large epithelial-like cells. (B) Flow cytometric analyses of magnetic and non-magnetic fractions from untreated or plasma-treated microparticles that are unconjugated, NR-conjugated or NB-conjugated. NR fluorescent signal is observed at FL2 (575 nm) and FL6 (660 nm). NB fluorescence is detected at FL6 (660 nm). All magnetic fractions had much greater side scatter (SSC) than the non-magnetic fraction indicating that the magnetically selected cells are more granular due to the presence of internalized microparticles. In the adrenal gland, the non-magnetic fraction cells are slightly larger than the cells of the magnetic fraction though these findings are only statistically significant for the plasma-treated samples. FSC indicates forward scatter or size. (C) Flow cytometric analyses of NR FL2 (575 nm) and FL6 (660 nm) and NB FL6 (660 nm) fluorescence in magnetic and non-magnetic fractions from rat liver single cell suspensions incubated with untreated or plasma-treated MPs again shows greater NR and NB FL6 (660 nm) signal compared to the non-magnetic fraction. All magnetic fractions had much greater side scatter (SSC) than the non-magnetic fraction. In the liver, the magnetic fraction cells are larger than the non-magnetic fraction. FSC indicates forward scatter or size. (D) SSC MFI from the magnetic fractions of different microparticle treatments and conjugations. Data shown is MFI for 3 independent experiments with SD. Black columns indicate magnetic fraction and light grey indicates non-magnetic fraction. NS=no statistically significantly difference, *p value<=0.05, **p value<=0.01, ***p<=0.001 as determined by two-way ANOVA using GraphPad Prism.

FIG. 9: Fluorescent signal of plasma-polymerised nanoparticles (PPN) conjugated to Nile Red or Nile Blue as assessed using a flow cytometer.

FIG. 10: Mean fluorescent intensity of Nile Blue-conjugated PPN as assessed in FL6 under different conditions. Nile Blue fluorescence increases with temperature and detergent washing.

FIG. 11: Nile red-conjugated PPN enter live mammalian cells and fluorescent more discretely in lipid droplets. 3T3-L1 pre-adipocytes were cultured for 30 mins in the presence of Nile red alone (left panels) or Nile Red-conjugated PPN (right panels) and imaged using an upright fluorescent microscope. Nile Red-PPN can enter cells and stain lipid droplets in a more discrete fashion compared to Nile Red alone.

FIG. 12: Mitotracker Green-conjugated PPN can cross cell membrane and enter mitochondria. Human breast cancer cells were incubated with Mitotracker Green-conjugated PPN and then imaged using an upright fluorescent microscope. Mitotracker Green-conjugated PPN have localised in cytoplasmic structures smaller than typical nuclei with the characteristic size, shape and location of mitochondria. Right panel is zoomed in image from centre of left panel.

FIG. 13: Glucose-iridium probe of glucose transport can be conjugated to PPN and enter cells. Left panel, increasing concentrations of glucose-iridium probe incubation with PPN leads to increased fluorescent signal in fluorescent channel 10. Right panel, live mouse brown adipocytes were incubated with glucose-iridium complex alone (middle peak) or with PPN conjugated to the glucose-iridium probe (bottom peak). The top peak is unstained cells control. The increased fluoresce in the cells in the orange peak indicates entry and fluorescence of the PPN-conjugated to the glucose-iridium probe.

FIG. 14: PPN lipophilicity assay. Six billion PPN were resuspended in double distilled water (50 μL) and then mixed with 500 μL diluted Nile red (top panel) or Nile Blue (lower panel). Following 30 mins of conjugation, PPN were washed of unbound Nile Red or Nile Blue, resuspended in double distilled water and then an equal amount of 1-Octanol was added. Tubes were vigorously shaken and images taken after 10 mins when the two layers had partitioned. Nile red-conjugated PPN clearly partition to the upper 1-Octanol phase. Nile Blue-conjugated PPN show a similar partitioning. These observations suggest that the covalent conjugation of lipophilic probes does enhance the lipophilicity of PPNs potentially aiding transport across the lipid bilayer.

FIG. 15: Nile Red conjugated PPN, Nile Blue conjugated PPN, Nile Red conjugated plasma-treated microparticles, and Nile Red conjugated plasma-treated microparticles can cross the complex microalgal cell wall, as shown in N. occulata.

FIG. 16: Nile Red conjugated PPN, Nile Blue conjugated PPN, Nile Red conjugated plasma-treated microparticles, and Nile Red conjugated plasma-treated microparticles can cross the complex microalgal cell wall, as shown in P. tricornutum.

FIG. 17: Spectral scan showing that Nile Red conjugated PPN can carry beta-cyclodextrin.

### DEFINITIONS

In describing and claiming the present invention, the following terminology has been used in accordance with the definitions set out below. It is also to be understood that the terminology used herein is for the purpose of describing particular embodiments of the invention only and is not intended to be limiting. Unless defined otherwise, all technical and scientific terms used herein have the same meaning as commonly understood by one having ordinary skill in the art to which the invention pertains.

As used herein the term “about” can mean within 1 or more standard deviation per the practice in the art. Alternatively, “about” can mean a range of up to 20%. When particular values are provided in the specification and claims the meaning of “about” should be assumed to be within an acceptable error range for that particular value.

In the context of the present invention, the words “comprise”, “comprising” and the like are to be construed in their inclusive, as opposed to their exclusive, sense, that is in the sense of “including, but not limited to”.

The terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

Other than in the operating examples, or where otherwise indicated, all numbers expressing quantities of ingredients or reaction conditions used herein are to be understood as modified in all instances by the term ‘about’.

The recitation of a numerical range using endpoints includes all numbers subsumed within that range (e.g., 1 to 5 includes 1, 1.5, 2, 2.75, 3, 3.80, 4, 5, etc.).

In the context of the present invention, the term “particle” means a substance engineered from plasma, plastics, ceramics, glass, silca, polymers, minerals, inorganic salts, carbon, metals, or composites thereof. Particles are characterised by their size with the term “microparticle” meaning a particle with a diameter of between 0.1 μm and 200 μm, and the term “nanoparticle” meaning a particle with a diameter of between 1 nm and 100 nm. The term “particle” is intended to have the material science definition and does not encompass exosomes, macrovesicles, apoptotic bodies and the like.

The term “cell permeant agent” means a substance that is able to pass through or migrate across a cell membrane.

The term “fluorescing” means to produce, undergo, or exhibit fluorescence or luminescence.

The term “payload” means a substance to be carried by the a plasma-treated particle or plasma-synthesised particle into a cell.

The term “paramagnetic particle” means a particle that can be manipulated using a magnetic field. Such particles are also referred to as magnetic beads or magnetic microparticles. In some embodiments, the paramagnetic particle of microparticle contains a paramagnetic (e.g., an iron oxide) core which is encapsulated by a plastic (e.g., polystyrene) shell or coating. Alternatively, the paramagnetic particle contains a plastic core that is encapsulated or impregnated by a paramagnetic substance.

### PREFERRED EMBODIMENT OF THE INVENTION

Although the invention has been described with reference to certain embodiments detailed herein, other embodiments can achieve the same or similar results. Variations and modifications of the invention will be obvious to those skilled in the art and the invention is intended to cover all such modifications and equivalents.

Delivery of particles to cells is currently dependent on uptake via endocytosis or via targeting of cell surface markers. However, endosomal entrapment and subsequent degradation in acidic compartments of the endo/lysosomal pathway means that cargo molecules (e.g., proteins, peptides, carbohydrates, nucleic acids, small molecules or therapeutic agents) bound to the particles may not reach intracellular locations intact. Additionally, current magnetic activated cell sorting is limited to isolating cells according to cell surface markers/characteristics.

The invention presented here is a novel system for generating particles for use in delivering a payload to intracellular locations or to isolate cells using magnetic activated cell sorting according to intracellular cytological characteristics or traits.

Plasma treatment results in two profound changes in the surface of the particles. Firstly, the particles exhibit an unexpected increase in lipophilicity, enabling transport across mammalian cell membranes. Secondly, radicals formed on the particle surface from ion implantation can covalently bind (a) cell permeant agents that also binds to a cellular component of interest or (b) payloads (e.g., proteins, peptides, carbohydrates, nucleic acids, small molecules or therapeutic agents) for delivery to intracellular locations.

Using the present invention, intracellular characteristics, such as lipids, organelles, structures, biomolecules, etc., can now be used as targets for intracellular magnetic cell sorting. Previously, access to intracellular markers requires fixation and permeabilisation of cells, which kills them. In the present invention, magnetic particles can enter live cells and bind to a cellular component of interest, allowing for magnetic cell sorting of the cell based on intracellular characteristics. This allows isolation of previously inaccessible cell types by magnetic cell sorting, such as cells with high lipid content, active mitochondria, specific organelles or cytoskeletal structures or other lineage-or cell type-specific intracellular traits.

Further, the present invention also enables the intact delivery of payloads (e.g., proteins, peptides, carbohydrates, nucleic acids, small molecules or therapeutic agents) to intracellular locations by bypassing the endo/lysosomal pathway.

The present invention is further described by the following non-limiting examples.

### EXAMPLES

### Plasma Immersion Ion Implantation (PIII) Treatment of Magnetic Particles

Paramagnetic microparticles were purchased from Spherotech InC (Lake Forest, IL USA; Cat #PMS-20-10) and are monodispersed smooth surface magnetite-coated polystyrene core particles (nominal diameter of 2.0-2.9 mm) encapsulated by a thick layer of polystyrene in water solution. Microparticles were separated from water using a magnet, washed twice with ddH₂O water, resuspended in 10 ml ddH₂O, snap-frozen in LN₂ and freeze-dried for 24 hours using an Alpha 2-4 LSCbasic freeze dryer (Christ, Osterode am Harz, Germany) to obtain dry magnetic particles.

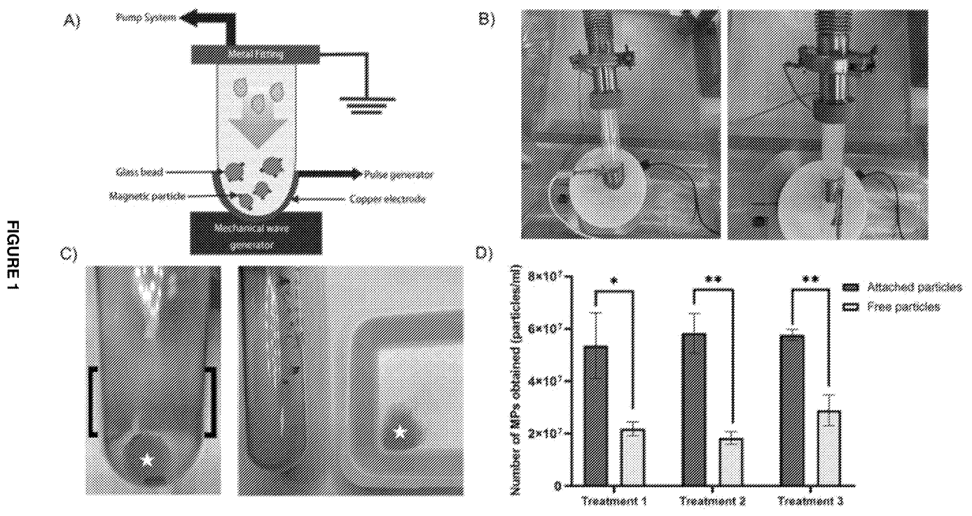

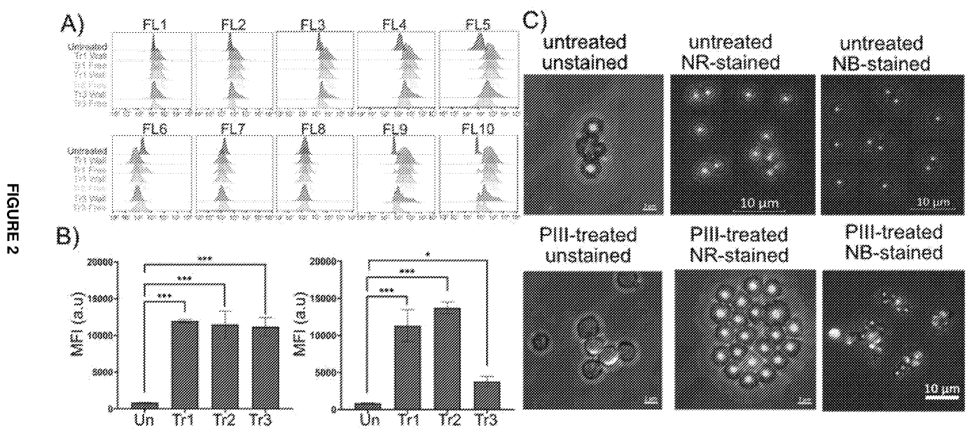

FIG. 1A shows the plasma apparatus schematic diagram consisting of a glass tube connecting to a pumping system (a rotatory pump and a turbo pump) via a metal KF fitting which acts as an electrode. A mechanical wave generator was used to vibrate the particles inside the glass tube. Approximately 8 mg of magnetic polystyrene microparticles were mixed with 240 mg of glass beads (425-600 μm, Sigma Aldrich) in a glass tube (length I=15 cm and diameter D=3 cm). The glass beads acted as carriers, rotating during the plasma treatment and thus enhancing the exposure of the microparticle surfaces to ion bombardment. The system was evacuated to a base pressure of 2×10-4 Torr before nitrogen gas was introduced to the tube with a flow rate of 20 sccm. The glass tube was then isolated from the pumping system and the pressure inside the tube was regulated to 350 mTorr for plasma treatment. A copper electrode was placed at the bottom of the glass tube to act as a cathode while the metal KF fitting was connected to earth. Negative voltage pulses of 7 kV, with a frequency of 3000 Hz and a pulse length of 40 μs were then applied to the copper electrode using a RUP 6 pulse generator (GBS Elektronik GMPH, Dresden, Germany). When the pulses were applied, nitrogen molecules were ionized, forming electrons and positive ions. The positive ions were accelerated under the electric field towards the copper electrode where the microparticles were shaken at 42 Hz frequency by the wave generator. Due to outgassing during plasma treatment and the isolation from the pump, pressure inside the tube slowly increased. Therefore, after one minute, the tube was reconnected to the pumping system to return to the base pressure before being refilled with nitrogen and repeating the 1-minute treatment cycle. The total treatment time was 10 minutes (10 cycles). The position of the copper electrode was varied either at the bottom of the tube (FIG. 1B left) or along the side of the tube (FIG. 1B right) to optimize the treatment effect. Three different regimes were compared, including Treatment 1(5 cycles with the electrode at the bottom and 5 cycles with the electrode along the side of the tube), Treatment 2 (10 cycles with the electrode along the side of the tube) and Treatment 3 (10 cycles with the electrode at the bottom of the tube).

PIII treatment led to a significant number of microparticles being attached to the wall of the glass tube while some microparticles collected at the bottom of the tube. These distinct microparticle populations were separated and referred to as ‘wall particles” or ‘free particles”, respectively (FIG. 1C). The two populations of MPs were suspended in ddH₂O at a concentration of 1 mg/mL. As the glass beads used in the ion bombardment are relatively heavy, these settled quickly at the bottom of the tube while MPs remained in suspension and could be separated. Microparticles were stored in amber vials at room temperature (RT) until use. The concentration of microparticles in solution was quantified using a hemocytometer.

Experiments were conducted where argon replaced nitrogen in the PIII process, with similar results.

### Immobilization of Fluorescent Probes to Plasma-Treated Magnetic Particles

Nile Red (Sigma Aldrich) was prepared in DMSO as a stock solution (100 μg/mL) and diluted to 1:10 in ddH₂O immediately prior to use. Diluted Nile Red (50 μL) was added to plasma-treated microparticles (10 μg per 50 μL ddH₂O) in 1.5 ml Eppendorf tubes, mixed well and incubated on a rocker for 1 hr. After the incubation, the plasma-treated microparticles were separated by placing Eppendorf tube on a magnetic separation rack (12320D, Invitrogen, USA) for 3 min, following by the removal of supernatant and resuspension of the particles with 1 ml ddH₂O. The washing step was repeated three times to remove unbound Nile Red.

To assess Nile Red and Nile Blue attachment, untreated and plasma-treated microparticles were further washed with 5% sodium dodecyl sulfate (SDS) and 2% Tween-20 in ddH₂O water at RT (25 °C) or at 70 °C for 30 min. After detergent treatment and magnetic separation, samples were washed with ddH₂O 3 times to remove remaining detergent. The microparticles were then resuspended in 100 μL PBS. To immobilize Nile Blue, untreated and plasma-treated microparticles (10 μg) were resuspended in 50 μL ddH2O, Nile Blue (Sigma Aldrich, 500 μmol in ddH₂O water) added at a ratio of 10 μL Nile Blue per 3 μg of microparticles, resuspended in 100 μL ddH₂O water, incubated for an hour and washed as described above. A stringent wash with detergent (5% SDS in ddH 2O water) at 70 °C for 30 min was conducted to test Nile Blue attachment. The attachment of Nile Red and Nile Blue to plasma-treated particles was analyzed using flow cytometry as described below.

### Zeta Potential and Dynamic Light Scattering Measurements

Zeta Potential and Dynamic Light Scattering measurements were conducted to investigate changes of surface charge and size of the plasma-treated microparticles after Nile Red and Nile Blue immobilization. The microparticles were conjugated to Nile Red or Nile Blue, and washed with SDS as described above. Approximately 200 mg of untreated and plasma-treated microparticles were conjugated and suspended in 1 ml of buffer solutions of various pH (pH3, pH4, pH7.4 and pH10) for the measurement. The buffers were prepared with a concentration of 10 mM and an ionic strength of 10 mM. The suspensions were added to a DTS1070 cell for zeta potential measurement and DTS0012 cell for size measurement. Both measurements were conducted using ZetaSizer Nano ZS (Malvern Instrument Ltd). Zeta potential was obtained from the electrophoretic mobility using the Smoluchowski equation. Three measurements were collected for each sample and the results for both treated and untreated particles are the average of 3 independent replicates.

### Lipophilicity Assessment

Microparticle lipophilicity was assessed using a modified version of the 1-octanol: water shake test (Wenlock et al., J Biomol Screen. 2011;16(3):348-55). This test is typically performed to assess the lipophilicity of pharmaceutical compounds and has not been reported for use with microparticles. An equal amount (250 μL) of ddH₂O and 1-octanol (Cat #472383, Sigma Aldrich) was added to a 1.5 mL Eppendorf tube, shaken and allowed to settle and partition into a 1-octanol phase on top and an aqueous phase on the bottom. Microparticles following plasma treatment, Nile Red or Nile Blue conjugation, or SDS wash were added to the 1-octanol: water mixture, shaken and allowed to settle and partition. Images were taken within 2 minutes of shaking. Fractions were then taken from each phase and the interface that formed in some treated samples and examined using a fluorescence microscope (Olympus BX53M with StreamMotion software). Brightfield and 540 nm fluorescence images were taken of the microparticles.

### Particle Fluorescence Analysis using a Becton Dickinson Gallios Flow Cytometer

To assess surface fluorescent signal, microparticles prepared as described above, were resuspended in 200 μL FACS buffer (0.05% w/v BSA in PBS) and analysed by Beckman Coulter Gallios Flow cytometer (Brea, CA, USA). At least 10,000 microparticles were analysed in each replicate, with samples from 3 independent experiments assessed. Microparticles conjugated with Nile Red were detected using the FL-2 and FL-6 channels while microparticles conjugated with Nile Blue were detected using the FL-6 channel only. Microparticle autofluorescence was detected in FL-9 and FL-10. Data were analyzed using the Kaluza G software package (London, UK).

### Cell Culture and Single Cell Suspension Preparation

Pre-adipocyte 3T3-L1 mouse embryonic fibroblasts (ATCC, Rockville, MD USA) were differentiated into mature lipid droplet-rich adipocytes as described in Assinder and Boumelhem (Mol Cell Endocrinol. 2021; 534:111381). 3T3-L1 cells were seeded at a density of 3000 cells/cm² and cultured in pre-adipocyte expansion media (DMEM: ThermoFisher Scientific) containing 10% (v/v) heat inactivated FBS (Scientifix), 1% (v/v) penicillin-streptomycin (ThermoFisher Scientific) in a humidified 5% CO₂ atmosphere at 37 °C. Cells were cultured until ~70% confluency was reached. Differentiation of 3T3-L1 to adipocytes was achieved according to ATCC protocol. Briefly, 3T3-L1 cells at 70% confluency were harvested by trypsinization, seeded at a density of 4×10⁴ cells/well in 12 well plates and grown to 100% confluency in pre-adipocyte expansion media. Cells were washed with PBS and cultured in differentiation medium (DMEM, 10% (v/v) heat inactivated FBS, 1 μM dexamethasone (Sigma-Aldrich), 0.5 mM methylisobutylxanthine (Sigma-Aldrich), 1 μg/mL insulin (ThermoFisher Scientific)) for 48 hours. Differentiation medium was then replaced with adipocyte maintenance medium (DMEM, containing 10% (v/v) heat inactivated FBS and 1 μg/mL insulin) and cells incubated for a further 10-14 days until adipocytes differentiated.

Adipocytes differentiated from 3T3-L1 cells were incubated with microparticles at 37 °C for 2.5 hours. Medium was aspirated and cells washed with PBS. Adipocytes were dissociated using TrypLE (ThermoFisher Scientific), washed with an equal volume of FACS buffer and centrifuged at 500×g for 5 min. The infranatant was discarded and buoyant, adipocyte fraction was mixed with the pelleted fraction which primarily consists of undifferentiated 3T3-L1 cells. Cells were then placed on a magnetic separation rack (12320D, Invitrogen, USA) for 3 min to collect the magnetically-selected cell fraction. The non-magnetic fraction was transferred to separate tubes while the magnetically selected fraction was resuspended in 700 μL FACS buffer for a second round of magnetic selection. Magnetic selection was conducted three times. Magnetically selected cells and non-magnetically selected cells were assessed by flow cytometry.

### Confocal Microscopy

In vitro 3T3-L1-derived adipocytes treated with microparticles in the cell culture plate were washed with PBS and fixed with 4% paraformaldehyde (Sigma Aldrich) for 15 min at RT. Fixed adipocytes were then washed three times with PBS and cover glass mounted with VectaShield antifade mounting media with DAPI (H2100, Vector Laboratories) and sealed with nail polish. Magnetically selected cells generated from 3T3-L1-derived adipocytes, cells were imaged fresh and not fixed to preserve cell morphology. Cell suspensions of magnetically selected and non-magnetically selected cells were resuspended in PBS and 50 μL pipetted onto a glass slide, mounted with a glass cover slip, sealed with nail polish and imaged immediately on a Zeiss LSM800 confocal microscope. This technique has been used previously to image single live cell suspensions of digested brown and white adipose tissue while preserving adipocyte morphology (Boumelhem et al., Adipocyte. 20176(2):112-123). Confocal images were taken on a Zeiss LSM800 confocal microscope (ACMM, University of Sydney) coupled to the Zen Blue software package. Nile Red, Nile Blue and DAPI were excited at 488 nm, 660 nm and 405 nm respectively and cells viewed by oil-immersed 40× or 63× objective.

### Preparation of Liver and Adrenal Tissue for Imaging and Flow Cytometry

Male Wistar rats (22-days old) were housed in filter top cages, kept under a 12-hour day-night cycle at constant temperature (21-22 °C) and provided food and water ad libitum. The welfare of the animals in the housing area and experiments conducted were in accordance with the Australian Code of Practice for the use of animals in research. Rats were killed by CO2 asphyxiation according to University of Sydney Animal Ethics Committee approval. Tissues were shared by Ms Mary Flokis and Prof Frank Lovicu following euthanasia for other purposes. Single cell suspensions of liver and adrenal glands were generated by mincing the tissues into fine pieces (~1-3 mm3) in FACS buffer containing 0.1% (w/v) Collagenase Type IV (Worthington) for 1 hour at 37 °C with vigorous shaking every 15 minutes. Digested tissue was further dispersed with repeated pipetting followed by filtration through a 350 μm polystyrene mesh. An equal volume of FACS buffer was added to the filtered single cell suspension to inactivate the collagenase and then centrifuged for 5 minutes at 500 g. Centrifugation pellets the stromal vascular fraction (SVF) while the buoyant fraction, containing adipocytes and lipid-containing cells, floats at the surface of the suspension. The infranatant was discarded and the buoyant, non-sedimented, adipocyte fraction were mixed with the pelleted fraction.

Single cell suspensions of digested liver and adrenal tissue were incubated with or without NR or NB conjugated plasma-treated microparticles at 37 °C for 1 hr. Phase contrast images were taken to visualize uptake of MPs. Images were taken at 32× magnification using a Zeiss Axiovert microscope equipped with the Zen Blue software package. Additional images were taken following magnetic selection to observe differences between fractions. Magnetic selection was conducted by placing cell suspensions on the magnetic separation rack for 3 min to collect magnetically selected cells. Supernatants were removed and kept in another Eppendorf tube and the magnetic selection fractions were resuspended in 700 μL FACS buffer. Cells from the magnetic and non-magnetic fractions were assessed by flow cytometry. Flow cytometry of magnetically selected, single cell suspensions of liver and adrenal tissue was performed on a Beckman Coulter Gallios Flow cytometer. Nile Red was detected using the FL-2 and FL-6 channels while MPs conjugated with Nile Blue were detected using the FL-6 channel only.

### Statistical Tests

Data obtained from flow cytometry was analysed using Flowjo (TreeStar, Ashland, OR, USA) and statistical analysis was preformed using Prism (GraphPad, USA). The results are expressed as mean±standard deviation (SD). Mean fluorescence intensity (MFI) at specific channels are compared using a two-way ANOVA. Differences in MFI between the magnetic and non-magnetic fractions were determined by Student's t-test or two-way ANOVA. A p value less than 0.05 was considered statistically significant.

### Plasma Immersion Ion Implantation Generates Two Populations of Magnetic Particles

Microparticles were placed in glass test tubes with glass beads to evenly distribute ion bombardment. FIG. 1A shows the design of the plasma treatment vessel. The effect of altering the electrode position was assessed by performing PIII treatment with the electrode either at the base of the glass tube (FIG. 1B left) for 5 cycles then along the side for another 5 cycles (Treatment 1); or 10 cycles with the electrode along the side of the tube (Treatment 2); or 10 cycles with the electrode at the base of the tube (Treatment 3) (FIG. 1B right). In performing PIII treatment in this way, it was observed that a significant number of particles attached to the wall of the glass tube while others collected at the bottom of the tube. Herein, these are referred to as “wall particles” or “free particles”, respectively. After PIII, the two populations of microparticles were separated and suspended in ddH₂O water for comparing the effect of plasma treatment (FIG. 1C). The frequency of the distinct magnetic particle types was assessed by counting using a hemocytometer (FIG. 1D). Approximately two-thirds of particles adhered to the wall while the remaining particles fell freely to the base of the glass tube.

### PIII-treated Magnetic Particles Exhibit Autofluorescence

Having identified two different types of microparticles following PIII treatment (wall particles and free particles), the ability of these different particle types to covalently conjugate the lipophilic probes Nile Red and Nile Blue was assessed using a flow cytometry protocol based on the Becton Dickinson Gallios 10-colour flow cytometer. The parameter settings were optimized using settings already established for cell-based flow cytometry with adjustments made to the voltages and gains for forward scatter (size), side scatter (granularity, light reflected at 90°) as well the fluorescent channels (FL) according to unstained and untreated microparticles.

Untreated and plasma-treated microparticles were assessed for autofluorescence across 10 FL channels (FIG. 2A). A moderate increase in autofluorescence was observed in fluorescent channels 2-5 (FL2 (575 nm), FL3, FL4 and FL5) (FIG. 2A). While untreated microparticles did not show significant autofluorescence, plasma treatment resulted in an increase in autofluorescence excited by the 405 nm laser (FIG. 2A) as observed in FL9 and FL10. All plasma-treated samples showed a statistically significant increase in FL10 autofluorescence (FIG. 2B). Free microparticles from Treatment 3 showed less autofluorescence than other samples suggesting that these may have received less ion implantation than the other samples (FIG. 2B). All wall particles showed high levels of FL10 autofluorescence suggesting similar PIII treatment. Surprisingly, the autofluorescent signal of FL6, (emission at 640 nm: excitation at 660 nm herein termed FL (660 nm)) was reduced in PIII-treated samples compared to untreated particles.

Confocal microscopy was performed on microparticles to assess the fluorescent signal following plasma treatment and following conjugation to Nile Red or Nile Blue. In FIG. 2C, the total fluorescence intensities are displayed on a pseudocolour scale. Yellow, cyan and magenta were chosen for pseudocolouring as these colours are more easily seen by all readers. Yellow represents fluorescence excited by the 405 nm laser, magenta (being closest in colour to the red of Nile Red) represents fluorescence excited by the 488 nm laser and cyan (being closest in colour to blue of Nile Blue) represent fluorescence excited by the 660 nm laser. The autofluorescent signal in the violet range can only be detected in the PIII-treated samples (yellow) and not in the untreated samples (FIG. 2C). Nile Red binding can be observed in both samples as magenta and Nile Blue can be observed as cyan. Images shown in FIG. 2C are merges of all three fluorescent channels with the brightfield images of microparticles.

### Covalent Attachment of Nile Red and Nile Blue to Magnetic Particles

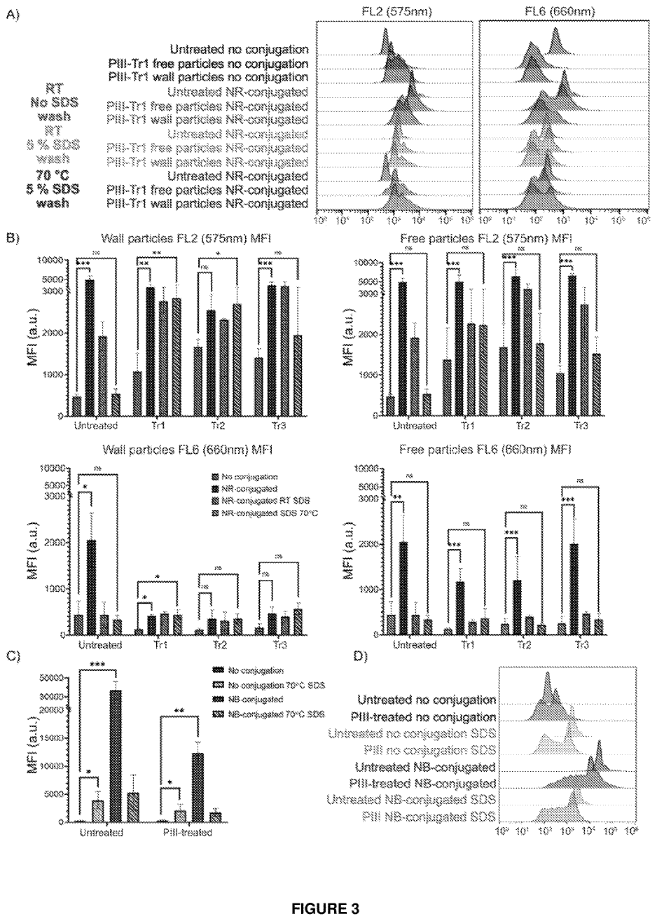

To assess covalent attachment, microparticles were treated with the three different PIII protocols, incubated with NR or NB and washed with ddH₂O or 5% SDS at RT or 70 °C (FIG. 3). Fluorescent signal in FL2 (575 nm) indicates NR fluorescence whereas signal in FL6 (660 nm) is indicative of NB fluorescence. NR bound to untreated microparticles and gave a bright FL2 (575 nm) fluorescent signal that was diminished by washing with SDS at 70 °C, to the level that there was no statistically significant difference between high temperature SDS wash and unstained samples (FIG. 3A and B). NR FL2 (575 nm) signal on wall particles but not free particles was preserved following high temperature SDS wash suggesting NR is covalently immobilized on plasma-treated wall particles and that the free particles are likely to have had less ion implantation. NR can red-shift according to the solvent and polarity of the local environment. Nile Red exhibited a red-shift when incubated with the commercially available polystyrene-coated microparticles used in this study. NR FL6 (660 nm) fluorescent signal was observed in all samples though the brightest signal was present on untreated microparticles without the high temperature SDS wash (FIG. 3B lower left panel). This is removed by washing with 5% SDS. As the wall particles from Treatment 1 were the only microparticles that showed a significant change in NR FL6 (660 nm) following high temperature SDS wash, indicative of the highest level of covalent immobilization, these particles were selected for use in subsequent studies.

Similar analyses were performed on NB-conjugated microparticles following washes to assess covalent attachment. In contrast to NR, the NB signal was also lost from PIII-treated microparticles with a high temperature wash (FIG. 3C and D). The NB FL6 (660 nm) signal of microparticles incubated with NB then washed with SDS gave the same signal as samples incubated with 5% SDS alone (FIG. 3C and D).

### Characterization of Plasma-Treated Magnetic Particles

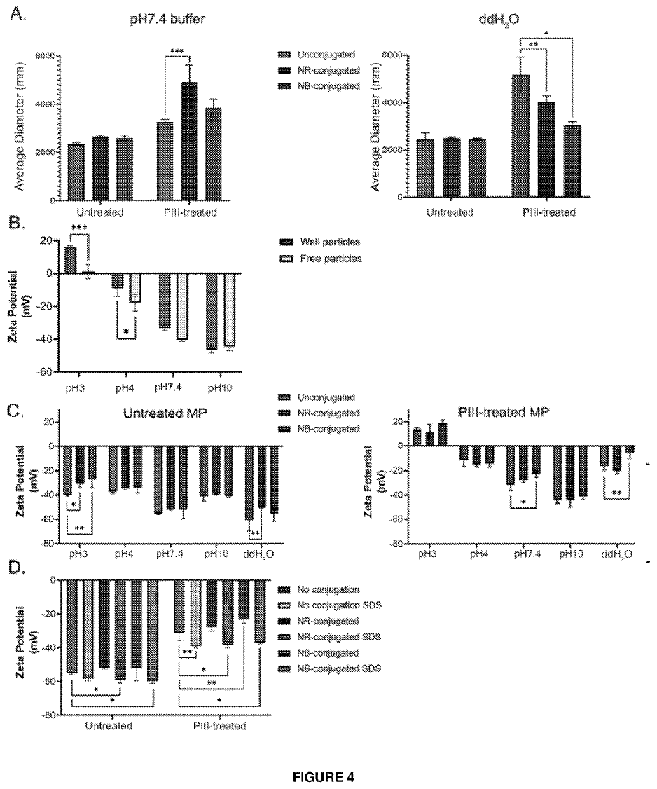

Dynamic light scattering analysis was performed to better understand the effects of the plasma treatment on commercially available magnetic microparticles and their conjugation with Nile Red and Nile Blue. As seen in FIG. 4A, in pH 7.4 buffer and in ddH₂O, untreated microparticles did not vary in size. PIII treatment and conjugation to Nile Red or Nile Blue did impact the dynamic light scattering results in a pH-dependent manner. At pH 7.4, unconjugated microparticles did not show a significant increase in size however in ddH₂O, the particle size was almost double suggesting that PIII-treated microparticles aggregate in distilled water (FIG. 4A). Nile Red conjugation led to an increase in size in PIII-treated microparticles in both pH7.4 buffer and ddH₂O whereas Nile Blue conjugation had a less significant impact on size. These data suggest that PIII treatment and Nile Red conjugation can alter the size of microparticles, presumably by inducing aggregation into clusters. This is indicative of Nile Red conjugation to the surface of the microparticles as Nile Red is hydrophobic and readily aggregates in aqueous solution.

Surface charge of microparticles following plasma treatment and probe conjugation was assessed by determining the zeta potential using a ZetaSizer Nano ZS (Malvern Panalytical). The zeta potential of the wall particles at low pH was more positive than that of the free particles and they were both more positive than untreated microparticles at the same pH (FIG. 4B). This is due to the incorporation of nitrogen containing groups due to the ion bombardment from nitrogen plasma which results in more nitrogen containing groups, such as amine groups, on the polymer surface. Amine groups, especially NH₂ groups, are easily protonated to form NH₃⁺ in acidic solution, leading to a more positive surface charge. The higher positive charge of the wall particles suggests that they were more thoroughly exposed to the plasma ion bombardment than the free particles.

Untreated microparticles showed statistically significant modifications in zeta potential at pH 3 (FIG. 4C) when conjugated to NR or NB suggesting that these probes did have an effect on surface charge. Changes in zeta potential were observed in the plasma-treated samples incubated in pH7.4 buffer and ddH₂O. The largest changes were observed in NB conjugated plasma-treated microparticles which showed a shift to positive charge in line with the cationic nature of Nile Blue. When untreated or plasma-treated microparticles were conjugated to Nile Red or Nile Blue and then washed with 5% SDS, the surface charge changed again towards the negative (FIG. 4D). This was more pronounced in the plasma-treated samples where all SDS-washed samples showed a shift towards negative surface charge following SDS wash (FIG. 4D). These data suggest that the hydrophobic tail of SDS molecules associates with lipophilic Nile Red or Nile Blue molecules attached to the microparticles through hydrophobic interactions, making their surface charge more negative.

### Assessment of Lipophilicity of PIII-Treated Magnetic Particles

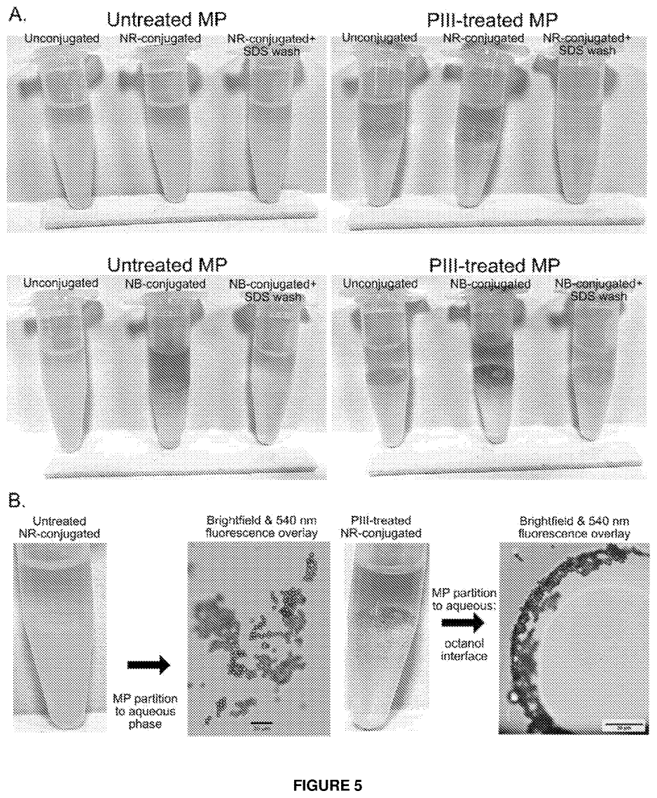

As Nile Red and Nile Blue are lipophilic dyes, the lipophilicity of the microparticles following plasma treatment and conjugation was assessed. Microparticles were added to Eppendorf tubes containing an equal mixture of 1-octanol and water and shaken to mix the liquids. Almost immediately after shaking, the 1-octanol partitioned to the top layer and the water to the bottom layer (FIG. 5A). While not a quantitative assay, this system rapidly reveals differences between MPs. The most immediately noticeable feature was that untreated microparticles partitioned to the aqueous phase while plasma-treated microparticles settled at the interface between the 1-octanol and water regardless of conjugation (FIG. 5A). The settlement of plasma-treated microparticles at the interface indicates that their surfaces have both hydrophilic and hydrophobic/lipophilic domains which can interact with both water and 1-octanol. A SDS wash has little effect on the partitioning of the untreated microparticles however SDS-washed Nile Red-conjugated plasma-treated microparticles remained in the 1-octanol phase longer than the same samples without SDS washing. While it typically took 1 min for the plasma-treated microparticles to settle at the interface, SDS washing led to them taking up to 10 mins to settle at the interface (FIG. 5A, top right panel). This is consistent with the attachment of some SDS molecules to the microparticle surfaces as the long hydrocarbon chain of the SDS molecule is hydrophobic and hence would have strong affinity for the 1-octanol phase.

A pink colour observed in the 1-octanol phase with both the untreated microparticles and plamsa-treated microparticles samples after Nile Red conjugation indicates that physisorbed Nile Red molecules were released into the 1-octanol due to the lipophilic nature of Nile Red and 1-octanol. More Nile Red molecules were associated with the plasma-treated microparticles as evidenced by pinker colour. This is consistent with the weaker fluorescence measured in flow cytometry for NR-conjugated plasma-treated microparticles relative to the NR conjugated untreated microparticles because the fluorescence of Nile Red is known to decrease when Nile Red forms aggregates. After SDS washing, most physisorbed Nile Red molecules were removed from both types of particles, resulting in a significant reduction of the pink colour observed in the 1-octanol phase for the SDS washed samples. The cloudiness of the 1-octanol phase (top layer, FIG. 5A, top right panel) for the NR conjugated plasma-treated microparticles relative to the untreated controls shows that a layer of NR molecules remains conjugated on the surface of the plasma-treated microparticles even after the SDS washing suggesting that this layer is covalently attached to the plasma-treated microparticles surfaces. In contrast, the fact that the untreated microparticles segregated to the water phase after Nile Red conjugation, both with and without SDS washing, indicates relatively weak Nile Red association on their surfaces.

NR-conjugated untreated microparticles were collected from the aqueous phase and examined using a fluorescent stereomicroscope (FIG. 5B, left panel). Overlay of the 540 nm fluorescence for Nile Red and the brightfield signal show no fluorescent signal is detectible. In contrast, Nile Red-conjugated plasma-treated microparticles collected from the interface, where they partitioned, showed intense Nile Red fluorescence and clustered around droplets of 1-octanol stained with Nile Red (FIG. 5B, right panel). These data agree well with the release of Nile Red in the 1-octanol phase (FIG. 5A), suggesting that the hydrophobic interaction with 1-octanol removed the lipophilic Nile Red that was physisorbed on the untreated MPs but could not remove the Nile Red covalently attached to the plasma-treated MPs.

A surprising result was that plasma-treatment alone results in profound changes in lipophilicity of the microparticles, which enhances the ability of the microparticle to move across cell membranes. As shown in FIG. 5A, untreated microparticles are found in the lower water layer but plasma-treated microparticle, regardless of presence or absence of Nile Red or Nile Blue, were found at the interface between the 1-octanol and water showing that there is a change in the lipophilicity of the surface of the microparticle. This is unexpected as plasma-treated flat surfaces usually become more hydrophobic and less lipophilic. Addition of the NR or NB increases lipophilicity further.

### Targeting of Nile Red- and Nile Blue-Conjugated Plasma-Treated Microparticlesto Live Adipocytes

Having confirmed that Nile Red is covalently attached to plasma-treated microparticles, these were next assessed for their targeting ability to enter cells and home to lipid droplets. In the case of Nile Blue-conjugated plasma-treated microparticles, covalent attachment could not be confirmed due to high SDS fluorescence but these particles were used for incubation with cells. To prevent contamination by bacteria or other micro-organisms during cell culture, NR conjugated untreated and plasma-treated microparticles and NB conjugated untreated and plasma-treated microparticles were UV-irradiated inside a biohazard laminar flow hood where all subsequent cell culture was performed. UV-irradiation (10 mins) had no impact on NR or NB fluorescence as assessed by flow cytometry. To assess uptake of NR conjugated untreated and plasma-treated microparticles and NB conjugated untreated and plasma-treated microparticles, 3T3-L1 fibroblasts were differentiated into lipid droplet-rich adipocytes in vitro. Once lipid droplets were observed in culture, NR conjugated untreated and plasma-treated microparticles and NB conjugated untreated and plasma-treated microparticles were added to the cultures with the ratio of 10 microparticles per cell. Cultures were monitored using a stereomicroscope with fluorescence light source suitable for detecting NR. After 2.5 hours of culture, cultures were found to have highly fluorescent lipid droplets in NR microparticle samples (both untreated and plasma-treated). Nile Red fluorescence increases in the presence of neutral lipids such as cholesterol and triglycerides. Hence the fluorescence observed in these droplets is from Nile Red changing to a more fluorescent state in the presence of these lipids.

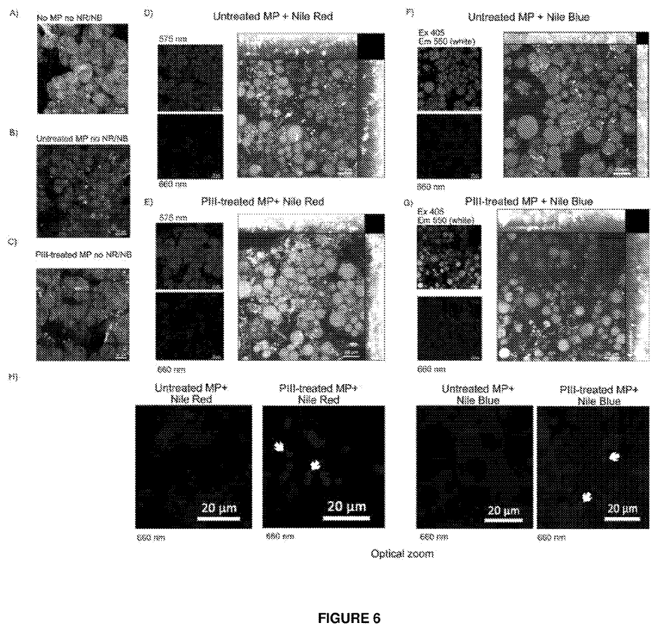

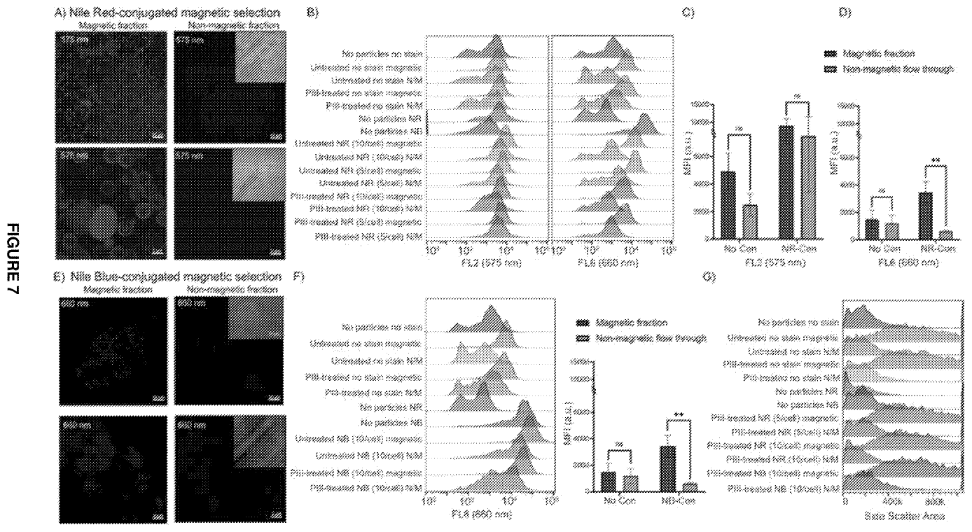

To obtain higher resolution understanding of the interactions between NR conjugated untreated and plasma-treated microparticles and NB conjugated untreated and plasma-treated microparticles with 3T3-L1 adipocytes, cultures were fixed and incubated with phalloidin and carborane coumarin, a lipophilic probe that also senses neutral lipids but emits in the violet range. Unconjugated microparticles showed no fluorescent signal following incubation of 3T3-L1 adipocytes and imaged by confocal microscopy (FIG. 6A, B and C) regardless of treatment regime. Both NR conjugated untreated and plasma-treated microparticles showed bright NR lipid droplet fluorescence indicating that microparticles bearing NRs had indeed entered the adipocytes and targeted the lipid droplets with NR then fluorescing in the presence of neutral lipids. Numerous NR conjugated plasma-treated microparticles can be detected in the adipocytes while fewer are observed in the adipocytes incubated with NR conjugated untreated microparticles (FIG. 6D, E, H).

Confocal imaging of NB microparticle adipocyte cultures revealed that both untreated and plasma-treated particles conjugated to NB generated a fluorescent signal which was detected in the cytosol of the adipocytes and did not co-localize with the carborane coumarin-stained lipid droplets (FIG. 6F, G). This is as expected as NB shows peak fluorescence in the presence of unsaturated free fatty acids which will be found in the cytosol and not the lipid droplets. While Nile Blue signal could be detected in the adipocytes incubated with untreated microparticles, the fluorescent signal was faint and diffuse (FIG. 6F). In contrast, the plasma-treated microparticles could be clearly observed in the cytoplasm of adipocytes (FIG. 6G). To better observe the NR signal, optical zoom images from panels D (NR conjugated untreated microparticles) and E (NR conjugated plasma-treated microparticles) are shown in FIG. 6H. Plasma treatment results in greater 660 nm fluorescence intensity. In contrast, the 660 nm fluorescence in the cells incubated with NR conjugated untreated microparticles is diffusely distributed throughout the adipocytes. A similar result was observed in the optical zoom images from NB-conjugated microparticle adipocyte incubations. Cells incubated with NB conjugated untreated microparticles showed very faint diffuse cellular staining while cells incubated with NB-conjugated plasma-treated microparticles showed discrete NB-bright structures approximately 2 μm in size (FIG. 6H).

### Histochemomagnetic Separation of 3t3-l1-Differentiated Adipocytes by Lipid Content Targeting

Following optimization of the manufacturing of NR conjugated plasma-treated microparticles and NB conjugated plasma-treated microparticles which can enter the cell and be trafficked to the appropriate location depending on the probe, it was investigated whether these particles could be used to isolate cells from a mixture according to lipid contents. To do so, 3T3-L1 fibroblasts were again differentiated into adipocytes, untreated microparticles, NR conjugated plasma-treated microparticles or NB conjugated plasma-treated microparticles added for 2.5 hours and then the cells were harvested by trypsinization and separated using a magnet. Cells from the different fractions were imaged by confocal microscopy. As can be seen (FIG. 7A, left panels), cells from the magnetic fraction contain large amounts of Nile Red-conjugated plasma-treated microparticles. In contrast, the cells from the non-magnetic fraction show very little fluorescent signal indicating few if any particles. This demonstrates that the magnetic fraction is made up of cells containing Nile Red conjugated plasma-treated microparticles.

Magnetic separation was performed 3 times to enrich for the magnetic and non-magnetic fractions (data shown is of the first cell separation step). Flow cytometry was performed using the probes on the particles themselves as indicators of the presence of either lipid droplets (NR) or free fatty acids (NB). Cells stained by the dyes themselves (without microparticles) were used to indicate free dye presence and also to assess any enrichment of positive populations by the magnetic selection. Cells isolated using NR conjugated plasma-treated microparticles in the magnetic fraction were compared to non-magnetic fraction and NR alone using 488 nm laser by flow cytometry (FIG. 7B). No significant difference was observed by Nile Red conjugated untreated and plasma-treated microparticles in FL2 (575 nm) (FIG. 7C). In contrast, when assessed using FL6 (660 nm) the magnetic fraction of both Nile Red conjugated untreated and plasma-treated microparticles showed a significant enrichment in fluorescent signal (FIG. 7D). The NR FL6 (660 nm) fluorescent signal of the magnetic fraction was 10-50 times brighter than the first non-magnetic population demonstrating enrichment of NR-positive lipid droplet-rich cells. These observations demonstrate that live lipid droplet-containing cells can be enriched by magnetic selection.

Nile Blue-conjugated plasma-treated microparticles were added to 3T3-L1 as described above. The cells were then subjected to three rounds of magnetic separation followed by imaging and flow cytometry. As can be seen (FIG. 7E, left panels), cells from the magnetic fraction contain large amounts of Nile Blue-conjugated particles with over 40 particles observed in some cells. In contrast, the cells from the non-magnetic fraction following Nile Blue conjugated plasma-treated particle separation show very little fluorescent signal indicating few if any particles. This demonstrates that the magnetic fraction is made up of cells containing Nile Blue conjugated plasma-conjugated microparticles. Again, the magnetic fraction had the highest NB FL6 (660 nm) fluorescent signal indicating that cells with free fatty acids were being separated from the mixture in the culture. The internalization of the microparticless is evident when comparing the side scatter, or light scattered at 90° which indicates the presence of more dense intracellular structures and is a known indicator of granularity. The magnetic fraction showed a strikingly higher side scatter (SSC) mean fluorescence intensity compared to the unstained and the non-magnetic fractions regardless of treatment or cargo.

### Histochemomagnetic Separation of Adrenal Gland and Adipose Cells Targeted Via Lipid Content

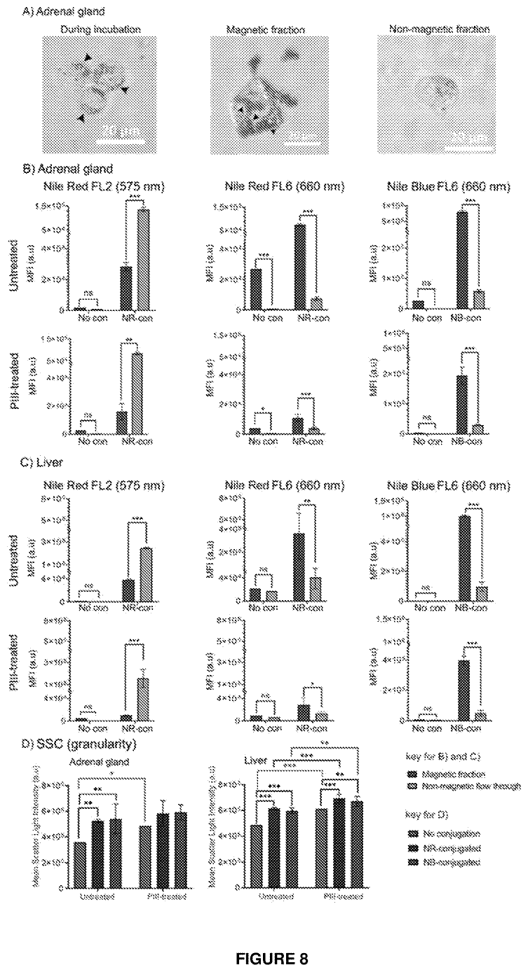

It was investigated whether this system could isolate lipid-rich cells from more complex single cell suspensions of organs. The liver is a site of lipid metabolism and storage while the adrenal glands are steroidogenic glands located above the kidney and show clear histological separation of neutral lipid-rich cortical cells whereas the adrenal medullary cells lack lipid droplets. Young male Wistar rat liver lobes and adrenal glands were dissected and dissociated in single cell suspensions which were then counted and incubated NR conjugated untreated and plasma-treated microparticles or NB conjugated untreated and plasma-treated microparticles. After 60 minutes of culture, particles could be observed internalized in the adrenal cell suspension (FIG. 8A “during incubation”). Following magnetic selection, the magnetic fraction contained cells with clearly observable microparticles present (FIG. 8A “magnetic fraction”). In contrast, the non-magnetic fraction contained cells with few if any particles and showed epithelial morphology (FIG. 8A). Red blood cells were also seen (data not shown).

Flow cytometric analysis of the magnetic and non-magnetic populations from NR-microparticle adrenal cell cultures showed a profound increase in NR FL6 (660 nm) fluorescence in the magnetic fraction compared to the non-magnetic fraction (FIG. 8B). Surprisingly, the cells in the non-magnetic fraction contained significantly higher NR FL2 (575 nm) fluorescence than the magnetic fraction suggesting that free Nile Red may be staining the non-magnetic fraction while the FL6 (660 nm) form of NR on the surface of the particles was found in the magnetic fraction. The magnetic fraction cells were approximately 4-fold brighter for NB fluorescence compared to the non-magnetic fraction (FIG. 8B).

Similar results were obtained with the incubation NR conjugated untreated and plasma-treated microparticles or NB conjugated untreated and plasma-treated microparticles with liver single cell suspensions (FIG. 8C). The NR FL6 (660 nm) signal was brighter in the magnetic fraction compared to the non-magnetic population (FIG. 8C). Again, the FL2 (575 nm) NR signal was brighter in the cells of the non-magnetic fraction suggesting staining with free NR. Similar to the adrenal gland observations, the magnetic fraction was approximately 4-fold brighter in FL6 (660 nm) than the non-magnetic fraction (FIG. 8C).

Having seen a significant increase in SSC signal in magnetic fractions from adipose cultures (FIG. 7), SSC in magnetic fractions from the adrenal gland and liver was assessed. In the adrenal gland magnetic fraction, a significant increase in SSC was observed in the cells isolated with microparticles that were plasma-treated and not conjugated to a dye compared to those that were untreated (FIG. 8D). This suggests that more plasma-treated microparticles may cross the cell membrane and be retained following magnetic selection possibly due to the increase in lipophilicity observed in FIG. 5. In the liver, the SSC was higher in the magnetic fraction from cells that were isolated using plasma-treated microparticles compared to untreated microparticles regardless of whether they were conjugated to NR or NB or unconjugated. These data suggest that plasma treatment and conjugation to the lipophilic probes Nile Red and Nile Blue may enhance the number of microparticles found inside the magnetically selected cells (FIG. 8D).

### Production of Nanoparticles by Plasma Polymerisation

Nanoparticles were synthesized via plasma polymerization in a stainless-steel chamber with a ring-like gas dispenser tube placed above the top electrode. The chamber was pumped down to the base pressure of <5×10⁻⁵ Torr to remove impurities and moisture. High purity argon, nitrogen and acetylene gases were supplied by BOC, Australia, and injected into the reaction chamber at set flow rates of 3, 10 and 6 sccms (standard cubic centimetres per minute) respectively. The mass flow rate of each gas was precisely regulated and monitored by computer software (FlowVision) and mass flow controllers (Allicat Scientific). Prior to nanoparticle generation, the pressure was set and maintained at a working pressure of 1.5×10⁻¹ Torr by regulating a mechanical valve that connects the chamber to the vacuum pumping system. The top electrode was powered by a 13.56 MHz (RF) generator and used to generate the electric field which creates plasma, triggering the polymerization process. To ensure the correct desired power is supplied to the reactor, a matching network was used to set the “forward power” and reduce the “reverse power” to 0 W.

### Immobilization of Fluorescent Probes to Plasma Polymerised Nanoparticles

Plasma polymerised nanoparticles (PPN) were incubated with Nile Red or Nile Blue. Nile Red (Sigma Aldrich) was prepared in DMSO as a stock solution (100 g/mL) and diluted to 1:10 in ddH₂O immediately prior to use. Nile Blue (Sigma Aldrich) was prepared in ddH₂O to a stock concentration of 5 mM and diluted 1:1000 in ddH₂O immediately prior to use. Diluted Nile Red or Nile Blue was added to nanoparticles (1×10⁶ nanoparticles in 50 μL of Nile Red or Nile Blue solution), mixed well and incubated on a rocker for 30 mins at RT (25 °C). After the incubation, nanoparticles were washed three times with ddH₂O to remove unbound Nile Red or Nile Blue. To assess Nile Red and Nile Blue covalent attachment, the nanoparticles were further washed with 5% sodium dodecyl sulfate (SDS) ddH₂O water at RT or at 95 °C for 30 min. After detergent treatment and centrifugation, samples were washed with ddH₂O, 3 times to remove any remaining detergent. Nanoparticles were resuspended in 100 μL PBS. The attachment of Nile Red and Nile Blue to the nanoparticles was analyzed using flow cytometry. Mitotracker Green was covalently attached to nanoparticles using the same incubation and wash protocol.

### Covalent Attachment of the Lipophilic Histochemical Dyes Nile Red and Nile Blue to PPN

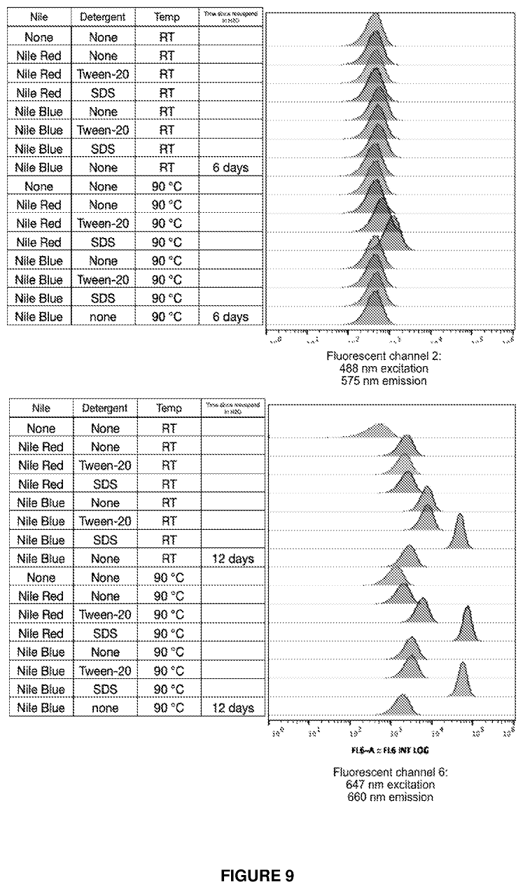

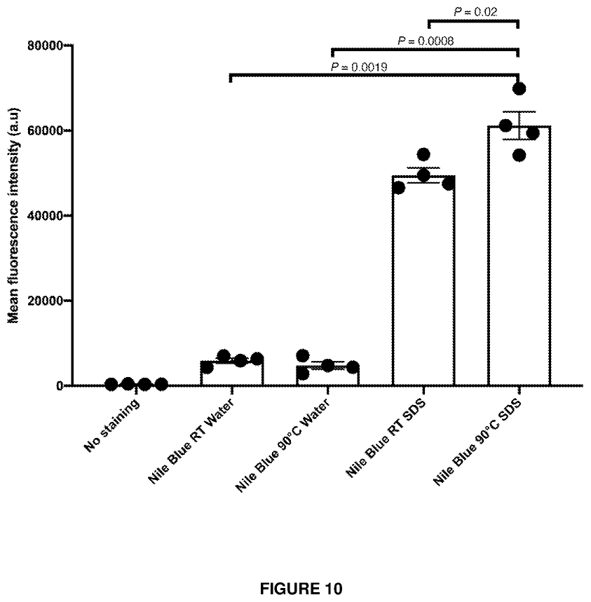

The lipophilic probes Nile Red and Nile Blue were incubated with PPN at RT for 30 mins and then washed with double distilled water (ddH₂O) or 5% sodium dodecylsulphate (SDS) at either RT or 90 °C in 1.5 mL tubes in a heat block. Flow cytometry was then performed on the nanoparticles with modified settings used on the Gallios 10-color flow cytometer to allow nanoparticles to be measured. As shown in FIG. 9, Nile red and Nile Blue both conjugated to PPN and both survived 30 mins of 5% SDS at 90 °C demonstrating covalent attachment. Both Nile red and Nile Blue showed brighter fluorescent signals on the PPN after heating and detergent wash. Interestingly, Nile Red showed a red-shift in fluorescence from the expected signal in FL2 to emission at FL6. This indicates the polarity of the surface of the nanoparticle as Nile red (but not Nile Blue) is solvatochromatic meaning fluorescence is dependent upon the polarity of the local environment (usually the solvent) that Nile Red finds itself in. FIG. 10 shows the mean fluorescent intensity of Nile Blue conjugated PPN as assessed in FL6 under different conditions. Nile Blue fluorescence increases with temperature and detergent washing demonstrating very strong (covalent) attachment of Nile Blue to the PPN.

### Nile Red-conjugated PPN Enter Live Cells and Label Appropriate Lipid Droplets

Nile red senses neutral lipids inside cells such as triglycerides and cholesterol. These are contained within organelles inside mammalian cells called lipid droplets. Nile red normally can cross the lipid bilayer cell membrane, move throughout the cytosol and home to the lipid droplets without requiring fixation as other lipophilic probes such as Oil Red O require.

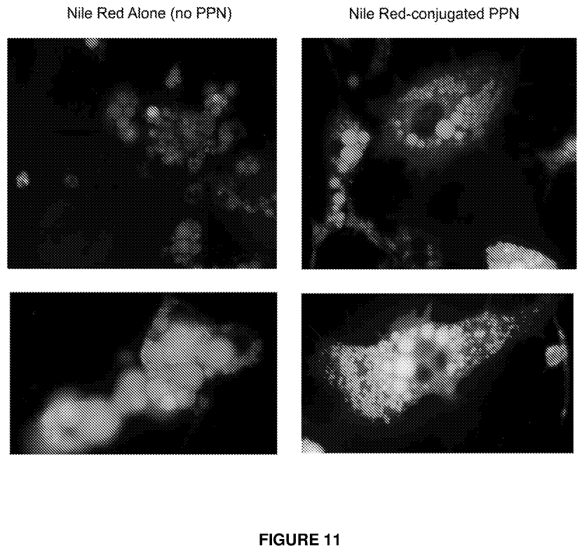

We assessed whether PPN conjugated covalently to Nile Red could enter cells in a similar fashion. Mouse 3T3-L1 cells were differentiated into adipocytes in vitro, incubated with Nile Red conjugated PPN. As can be seen in FIG. 11, Nile Red alone (without PPN) stains lipid droplets inside cells (red fluorescent signal, left panels). Nile Red conjugated PPN not only could cross the cell membrane without fixation but also homed to the lipid droplets (right panels). The fluorescent signal from Nile Red conjugated PPN was more discrete and focused compared to Nile Red alone suggesting that Nile Red conjugated PPN is a more specific and more intensely fluorescent histochemical probe offering improved signal: noise ratios.

### Conjugation of PPN to Mitotracker Green Leads to Targeting to Mitochondria

We next assessed whether other fluorescent probes could be conjugated to PPN and then direct the PPN across the cell membrane to the correct intracellular location. We chose the commercially available Mitotracker Green probe which homes to mitochondria, the powerhouse of the cell.

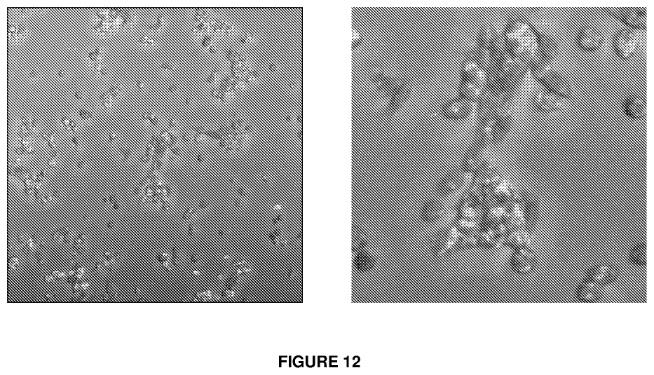

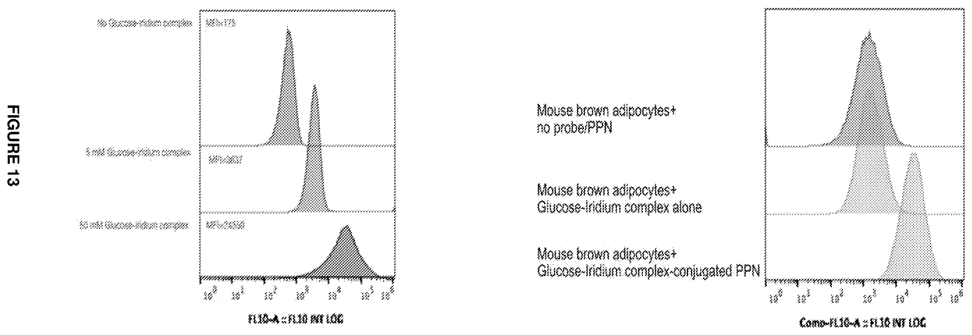

As can be seen in FIG. 12, fluorescent green signal is found within the cytoplasm of human breast cancer cells following incubation with PPN conjugated to Mitotracker Green. These fluorescent structures are the correct size and in the correct location for mitochondria, and found inside the cells without fixation or permeabilisation, suggesting that Mitotracker Green conjugated PPN can cross not only the cell membrane but also enter mitochondria.

### Fluorescent Glucose-Iridium Probes of Glucose Transport can Conjugate to PPN, Fluoresce and be taken up by Live Cells

To continue the analysis, we asked whether a previously published fluorescent probe of glucose transport (here termed Glucose-Iridium complex; Law et al., Inorg. Chem. 2013, 52, 22, 13029-13041) can bind to PPN and get inside cells. This probe fluoresces in the violet range, distinct from probes tested previously. As shown in FIG. 13, the glucose-iridium complex could be conjugated to PPN and fluoresce in the appropriate fluorescent channel (FL10: excitation 405 nm). Glucose-Iridium complex conjugated PPN could cross the live cell membrane of mouse brown adipocytes and be retained within the cytosol with the fluorescence detectible by flow cytometry (FIG. 13, right panel).

### Assessing Lipophilicity of PPN Conjugated to Lipophilic Probes

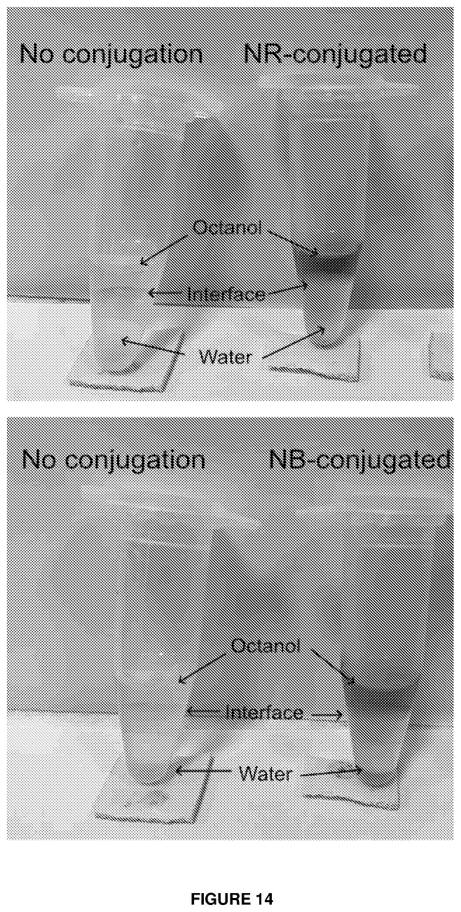

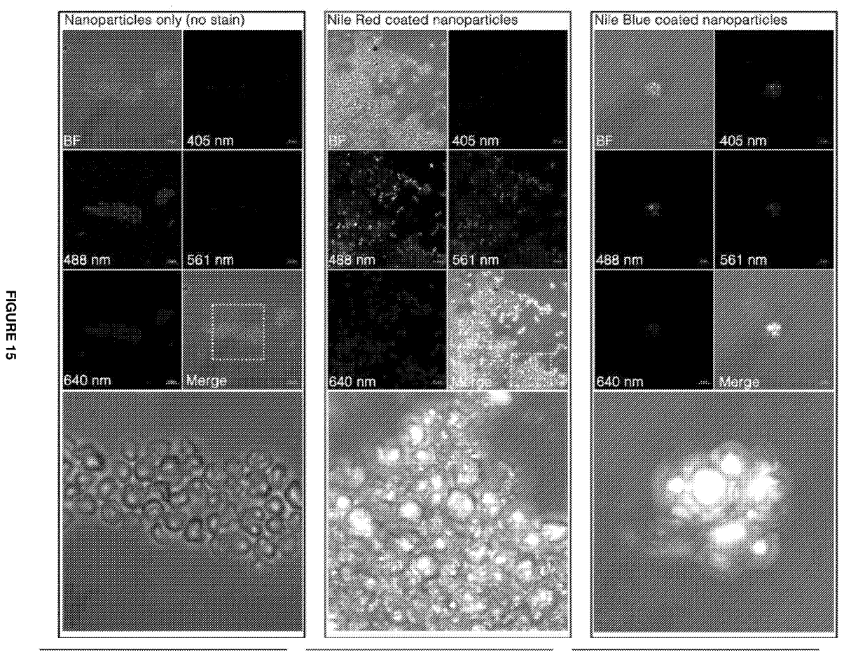

To determine whether the covalent conjugation of Nile Red or Nile Blue to PPN alters lipophilicity, and potentially entry into cells, 50 μL of PPN (approx 6 billion PPN) were incubated in 500 μL of Nile Red or Nile Blue for 30 mins on a rocker, washed to remove unbound probe then added to a tube where 500 μL of distilled water and 500 μL of 1-Octanol was added. The tubes were vigourously shaken and the two layers (heavier water on the bottom, lighter 1-Octanol on the top layer) rapidly formed. As can be seen in FIG. 6, PPN which had not been conjugated, collected at the interface between water and 1-Octanol as a brown-ish layer. In contrast, Nile red, presumably covalently bound to the PPN was found throughout the 1-Octanol layer suggesting enhanced lipophilicity when Nile red is bound to the PPN. Similar results are shown in the lower panel of FIG. 14 where Nile Blue-conjugated PPN are shaken in a mixture of water and 1-Octanol and the upper 1-Octanol layer clearly has blue signal present. These data suggest covalent attachment of lipophilic probes to PPN does indeed increase the lipophilicity of the particles.

### Nile Red and Nile Blue Conjugated Plasma Treated Magnetic Beads can Pass Through the Cell Wall of P. tricornutum and N. occulata

Microalgae, small plant-like organisms that make up a considerable portion of the world's plankton, have rigid cell walls that are challenging to penetrate in contrast to the fat molecules that make the mammalian cell membrane. It was assessed whether plasma-polymerised nanoparticles (PPN) or plasma-treated microparticles (PTMs) can enter microalgal cells as they can mammalian cells.

1 mL of DDH2O was repeatedly pipetted into a single well of a 12-well plate containing PPNs or PTMs. 1 μL of the solution was pipetted into a 1.7 mL Eppendorf tube. 10 μL of a 100 ng. mL−1 Nile Red solution or 5 mM Nile Blue solution was repeatedly pipetted to mix the PPNs or PTMs with the Nile dyes. The radicals in the surface of PPNs and PTMs bind to Nile Red or Nile Blue covalently. The solution was left to incubate for 30 minutes, in the dark at 25 °C. 1 mL of DDH₂O was added and the solution transferred to a 15 mL Corning tube for centrifugation. A benchtop centrifuge with capacity to centrifuge 1.7 mL tubes was not available. The solution was centrifuged at 4500 rpm for 15 minutes. The supernatant was discarded and Nile dye conjugated PPNs or PTMMs resuspended in 100 μL of PBS.

N. occulata, a small diatom species, or the larger P. tricornutum were centrifuged for 10 minutes at 2500 rpm. The supernatant was discarded, and cell pellet resuspended in 5 mL PBS. 100 μL of cells were pipetted into a 1.7 mL tube and mixed with 100 μL of:
PBS alone
Nile Red in PBS
Nile Blue in PBS
Nile Red and Nile Blue in PBS
PPNs in PBS
Nile Red conjugated PPNs in PBS
Nile Blue conjugated PPNs in PBS
PTMs in PBS
Nile Red conjugated PTMs in PBS
Nile Blue conjugated PTMs in PBS
Nile Red and Nile Blue conjugated PTMs in PBS

Samples were left to incubate for 1 hour, in the dark at 25 °C. Following incubation, samples were washed with 1 mL of PBS and centrifuged for 10 minutes at 2500 rpm. The supernatant was discarded, and cell pellet resuspended in 100 μL of PBS. 20 μL of the solution was pipetted onto a microscope slide and sealed with a coverslip.

A Zeiss LSM800 confocal microscope was used to image stained cells (unless noted, all images were taken using a 63× objective). The 405 nm laser was used to observe autofluorescence of PPNs and PTMMs. The 488 nm laser was used to observe autofluorescence of algae cells. The 561 nm laser was used to observe Nile Red staining. The 641 nm laser was used to observe Nile Blue staining. Unstained PPN or PTMs were used as controls.

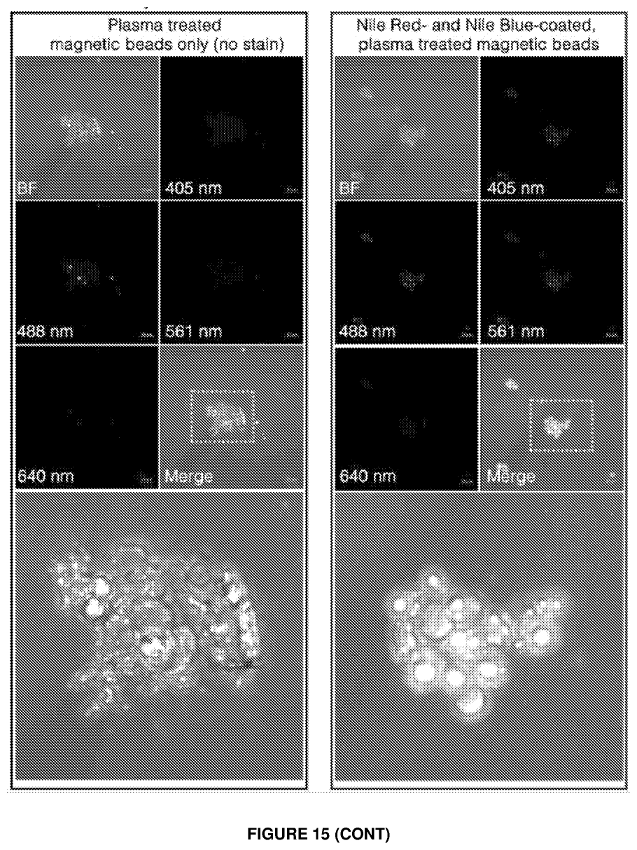

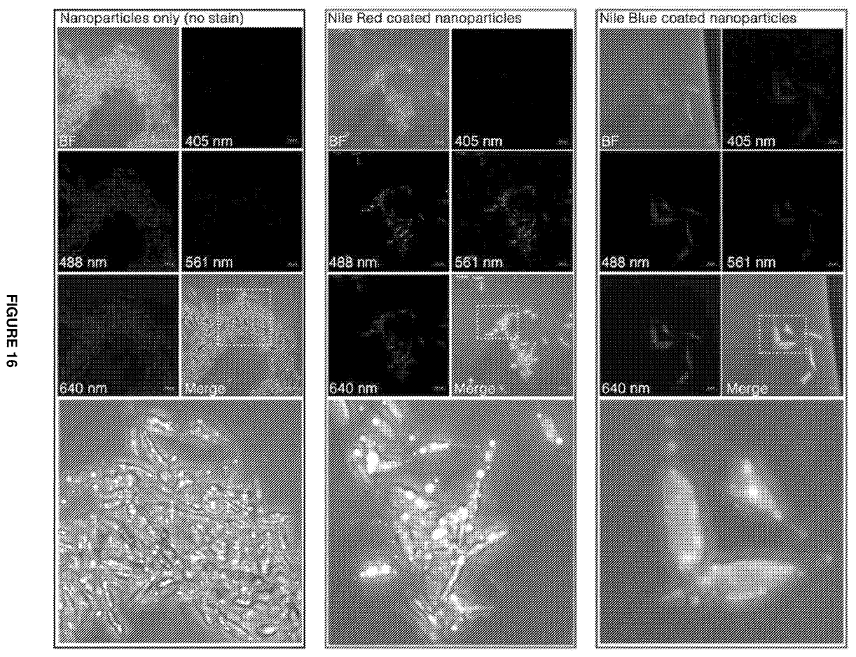

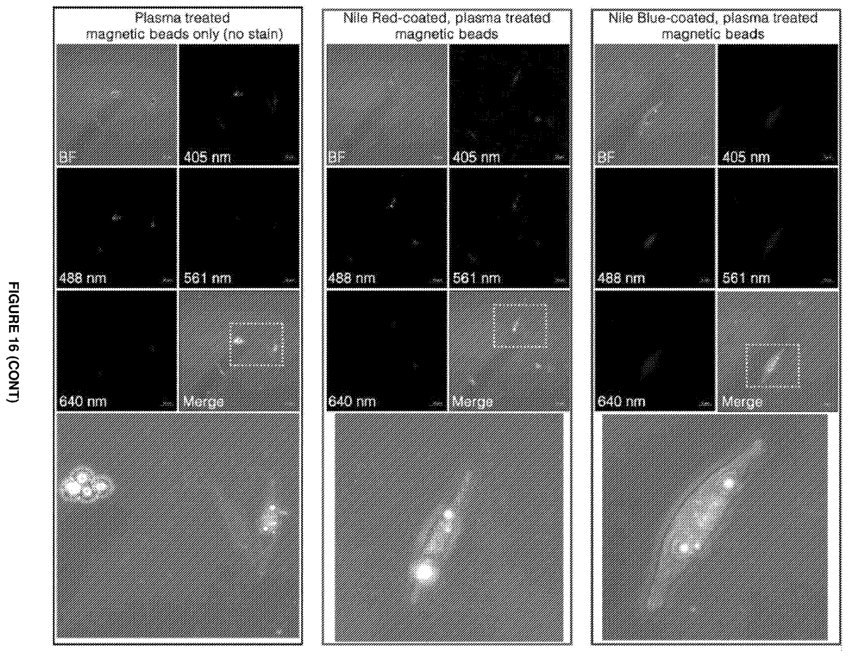

The results for N. occulata are shown in FIG. 15 and the results for P. tricornutum are shown in FIG. 16.

When microalgal cells were incubated with PPN or PTMs conjugated to Nile Red, bright magenta fluorescence was seen inside the cells. The amount seen inside cells incubated with PTMs with Nile Red is less than the Nile Red-conjugated PPN. This is because the PTMs are 10-times the size of the PPN and only one or two can enter the small microalgal cells. In contrast, a significant amount of Nile Red-conjugated PPN are found inside both microalgal species. Similar results were obtained with PPN or PTMs conjugated to Nile Blue, which binds to different lipids than Nile Red.

These results show that PPN or PTMs conjugated to fluorescent cell permeant dyes can readily cross the complex microalgal cell wall and lipid bilayer cell membrane. Such PPN or PTMs can be used to introduce DNA or RNA in microalgae or for magnetic selection of microalgae. This selection could be of use to obtain commercially important sub-strains of microalgae capable of producing greater amounts of useful biomolecules such as complex lipids (fat molecules) including omega-3, omega-6 and omega-9 oils.

### Using Nile Red Conjugated PPN to Carry Beta-cyclodextrin

In some people, lipids accumulate abnormally in very high levels due to defects in enzymes that process lipids. One possible therapy is beta-cyclodextrin (bCD), which forms tunnel-like structures which possess hydrophilic outer surfaces and lipophilic inner surfaces. Lipids are shuffled along the bCD tunnel and re-distributed from the droplet or cell. However, bCD is challenging to transport around the body and to the site of the lipid accumulation.

Nile Red (NR) was simultaneously covalently bound to PPN with bCD with and without washes in SDS (to remove NR and bCD not covalently bound) and a spectral scan of fluorescence emission was conducted for the following:
PPN
NR
PPN+NR
PPN+NR (SDS washed)
PPN+NR+bCD
PPN+NR+bCD (SDS washed)

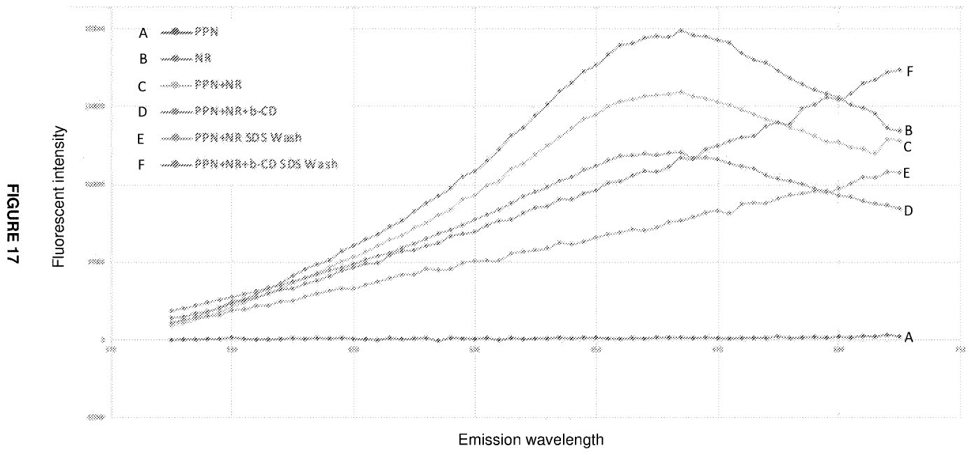

As shown in FIG. 17, SDS washing of PPN+NR reduced fluorescence compared to unwashed PPN+NR as aggregated NR has been washed off and leaving NR that is covalently bound. The fluorescence for PPN+NR+bCD was lower than that for PPN+NR as the amount of surface area that is available to NR is reduced by the attachment of bCD. SDS washing of PPN+NR+bCD increased fluorescence compared to unwashed PPN+NR+bCD, indicating that bCD is shielding NR from being washed off. This suggests that bCD is covalently attached and is functional. The emission spectrum of both SDS washed samples is different from water washed samples and are straight lines rather than curves. This reflects NR's red-shift in fluorescence in the presence of the change of polarity resulting from the SDS. These results demonstrate that NR and bCD are both covalently bound, and that NR conjugated PPN can be used to deliver therapeutic agents to intracellular locations.

1. A plasma-treated particle or plasma-synthesised particle to which a cell permeant agent is bound.

2. The particle according to claim 1, wherein the cell permeant agent is covalently bound to the particle.

3. The particle according to claim 1, wherein the cell permeant agent binds to a cellular component of interest.

4. The particle according to claim 1, wherein the cell permeant agent is lipophilic.

5. The particle according to claim 1, wherein the cell permeant agent fluoresces.

6. The particle according to claim 1, wherein the agent is selected from the group consisting of Nile Red, Nile Blue, Mitotracker Green, Glucose-Iridium complex and carborane coumarin, or analogues thereof.

7. The particle according to claim 1, wherein a payload is bound to the particle for delivery to a cell.

8. The particle according to claim 7, wherein the payload is selected from the group consisting of proteins, peptides, carbohydrates, nucleic acids, small molecules and therapeutic agents.

9. The particle according to claim 7, wherein the payload is covalently bound to the particle.

10. The particle according to claim 1, wherein the particle is treated or synthesised by plasma immersion ion implantation (PIII).

11. The particle according to claim 1, wherein the plasma-treated particle is a microparticle.

12. The particle according to claim 11, wherein the microparticle is a paramagnetic microparticle.

13. The particle according to claim 1, wherein the plasma-synthesised particle is a nanoparticle.

14. A method for producing a particle that is capable of migrating across a cell membrane, comprising treating a particle with plasma or synthesising a particle by plasma polymerisation, and binding a cell permeant agent to the particle.

15. A method for identifying a cell based on a cellular component of interest, the method comprising administering a plasma-treated particle or plasma-synthesised particle according to claim 1 to the cell, wherein the cell permeant agent binds to the cellular component of interest.

16. A method of delivering a payload to a cell, the method comprising administering a plasma-treated particle or plasma-synthesised particle according to claim 1 to the cell, wherein the payload is bound to the particle.

17. A method of magnetically sorting a cell based on a cellular component of interest, the method comprising:
administering a plasma-treated paramagnetic microparticle to the cell, wherein a cell permeant agent is bound to the microparticle, and wherein the cell permeant agent binds to the cellular component of interest; and
applying a magnetic field to the cell.
